(19) United States
(12) Patent Application Publication
(10) Pub. No.: US 2026/0070983 A1
(43) Pub. Date: Mar. 12, 2026

(54) PD-1 AGONIST ANTIBODIES AND METHODS OF TREATING AUTOIMMUNE DISEASES WITH A PD-1 AGONIST ANTIBODY

(71) Applicant: ELI LILLY AND COMPANY, Indianapolis, IN (US)

(72) Inventors: Yiqing FENG, Carmel, IN (US); Christina Marie KILEY, Indianapolis, IN (US); Ajay NIRULA, San Diego, CA (US); Jay Lawrence TUTTLE, San Marcos, CA (US); Evan Bo WANG, Carmel, IN (US); Pia Pauliina YACHI, San Diego, CA (US)

(21) Appl. No.: 19/104,548

(22) PCT Filed: Aug. 18, 2023
(86) PCT No.: PCT/US2023/072441

§ 371 Date: Feb. 18, 2025

Related U.S. Application Data
(60) Provisional application No. 63/399,391, filed on Aug. 19, 2022.
(60) Provisional application No. 63/401,355, filed on Aug. 26, 2022.
(60) Provisional application No. 63/488,903, filed on Mar. 7, 2023.

(51) Int. Cl. C07K 16/28; A61K 39/00; A61P 37/06

(52) U.S. Cl. CPC C07K 16/2818; A61P 37/06; A61K 2039/505; C07K 2317/33; C07K 2317/52; C07K 2317/565; C07K 2317/732; C07K 2317/75; C07K 2317/92

(57) ABSTRACT

The present disclosure relates to novel PD-1 agonist antibodies and methods and uses of anti-human PD-1 agonist antibodies for the treatment of autoinflammatory and/or autoimmune diseases. Also provided are doses and dosing regimens for the methods and uses of anti-human PD-1 agonist antibodies for the treatment of autoinflammatory and/or autoimmune diseases such as RA.

### SEQUENCE LISTING

The present application is being filed along with a Sequence Listing in ST.26 XML format. The Sequence Listing is provided as a file titled “30388 WO.xml” created 4 Aug. 2023 and is 48.8 kilobytes in size. The Sequence Listing information in the ST.26 XML format is incorporated herein by reference in its entirety.

### FIELD

The present disclosure generally relates to methods of treating autoinflammatory and/or autoimmune diseases, for example, rheumatoid arthritis (RA), with an antibody that binds to human programmed cell death protein 1, also known as human PD-1, and inhibits T cell activation, through agonism of the PD-1 pathway. More particularly, the present disclosure relates to methods of treating autoinflammatory and/or autoimmune diseases, for example, RA, with the PD-1 agonist monoclonal antibody, peresolimab.

### BACKGROUND

PD-1 and its ligands, PD-L1 and PD-L2, are important elements of the PD-1 pathway involved in immune homeostasis. There is evidence of defects in the PD-1 pathway having a significant pathophysiologic role in autoinflammatory and/or autoimmune diseases such as psoriasis (Gulati, et al. 2015), psoriatic arthritis (PsA) (see, for example, Bommarito, et al., 2017), vasculitis (see, for example, Zhang, et al., 2017), multiple sclerosis (MS) (see, for example, Trabattoni, et al., 2009), systemic lupus erythematosus (SLE) (see, for example, Mozaffarian, N., et al., 2008) systemic sclerosis (SSc) (see, for example, Fukasawa, et al., 2017), type 1 diabetes mellitus (T1DM) (see, for example, Guleria, I., et al., 2007) and RA (see, for example, Canavan, et al., 2021).

RA is an autoimmune disease characterized by chronic inflammation of synovial tissue, leading to destruction of the joint architecture. In some people, the condition can damage a wide variety of body systems, including the skin, eyes, lungs, heart, and blood vessels. The hallmark of the disease is a symmetric polyarthritis characteristically involving the small joints of the hands and feet. Systemic inflammation is characterized by laboratory abnormalities, such as anemia, elevated erythrocyte sedimentation rate, fibrinogen, and C-reactive protein (CRP) and by clinical symptoms of fatigue, weight loss, and muscle atrophy in affected joint areas. The presence of polyclonal high-titer rheumatoid factors and anticyclic citrullinated peptide (anti-CCP) antibodies provides evidence of immune dysregulation. It is recognized that elevated expression of PD-1 on T cells correlates with immune activation and disease activity in RA patients.

RA can negatively impact patients' ability to perform daily activities and may reduce the health-related quality of life. The main goal of treating RA currently is to reduce the signs and symptoms of the disease, prevent structural damage to bone and cartilage, and improve physical function from current state and social participation, thereby improving the health-related quality of life. The various therapeutic options available for managing RA and other autoinflammatory and/or autoimmune diseases include glucocorticoids and disease-modifying antirheumatic drugs (DMARDs), which include conventional synthetic DMARDs (csDMARDs), biologic DMARDs (bDMARDs), and targeted synthetic DMARDs (tsDMARDs). However, a significant number of RA and other autoinflammatory and/or autoimmune disease patients are inadequate responders, non-responders, or intolerant to such treatments, and the disease, including joint destruction in the case of RA, continues to progress despite the variety of the currently available treatments. In fact, it is thought that between 20-40% of RA patients do not respond to current treatments (see, for example, NCT05460832).

Despite reports of the PD-1 pathway having a significant pathophysiologic role in autoinflammatory and/or autoimmune diseases, intense interest in targeting this pathway for at least 15 years, and the generation of anti-PD-1 antibodies reported to be agonistic (see, for example, WO2019/168745 and WO2017/058859), the field has not yet provided safe and effective methods of using PD-1 agonistic antibodies to treat any disease, including autoinflammatory and/or autoimmune diseases.

Thus, there remains a crucial need for novel PD-1 agonist antibodies and/or improved treatment approaches, specifically in the form of dosage regimens for PD-1 agonist antibodies, which result in the stimulation of the physiological immune inhibitory pathway to restore immune regulation and that safely provide superior efficacy and/or durable efficacy in patients with autoinflammatory and/or autoimmune diseases, including in patients with moderately-to-severely active RA who have had an inadequate response to prior csDMARDs, bDMARDs and/or tsDMARDS.

### SUMMARY OF THE INVENTION

Accordingly, in a first aspect of the present disclosure, there is provided a method of treating autoinflammatory and/or autoimmune diseases comprising administering to a patient in need thereof an effective amount of an antibody that binds human PD-1 extracellular domain (ECD) with an affinity of between about 1 pM and about 100 nM as determined by Surface Plasmon Resonance (SPR) at 25° C., and that binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist.

In a second aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a heavy chain variable region (HCVR) and a light chain variable region (LCVR), wherein the HCVR comprises heavy chain complementarity determining regions (HCDRs) HCDR1, HCDR2, and HCDR3, and the LCVR comprises light chain complementarity determining regions (LCDRs) LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10.

In a third aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 1 pM to about 1 μM to Fc [symbol] RI;
   (ii) about 10 nM to about 10 μM to Fc [symbol] RIIA_131H;
   (iii) about 1 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist.

In another aspect, there is provided an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD, and wherein the antibody further comprises a human IgG1 Fc region variant selected from the group consisting of:
a) a human IgG1 Fc region variant comprising P247I and A339Q; and
b) a human IgG1 Fc region variant comprising S298A, E333A, and K334A;
c) a human IgG1 Fc region variant comprising S239D and I332E;
d) a human IgG1 Fc region variant comprising S239D, I332E, and A330L;
e) a human IgG1 Fc region variant comprising G236A, S239D, and I332E; and
f) a human IgG1 Fc region variant comprising G236A, S239D, I332E, and A330L; and wherein the antibody is a human PD-1 agonist.

In another aspect, the present disclosure provides an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and wherein the antibody further comprises a human IgG1 Fc region variant selected from the group consisting of:
   a) a human IgG1 Fc region variant comprising P247I and A339Q; and
   b) a human IgG1 Fc region variant comprising S298A, E333A, and K334A;
   c) a human IgG1 Fc region variant comprising S239D and I332E;
   d) a human IgG1 Fc region variant comprising S239D, I332E, and A330L;
   e) a human IgG1 Fc region variant comprising G236A, S239D, and I332E; and
   f) a human IgG1 Fc region variant comprising G236A, S239D, I332E, and A330L; and wherein the antibody is a human PD-1 agonist.

### BRIEF DESCRIPTION OF THE FIGURES

FIG. 1 illustrates the significantly greater improvement in participants treated with 700 mg peresolimab (LSM=−2.09; CI=−2.46, −1.72; p<0.001), and 300 mg peresolimab (LSM=−1.88; CI=−2.37, −1.38; p=0.017) administered intravenously (IV) Q4W versus placebo (LSM=−0.99; CI=−1.51, −0.47) in the primary efficacy outcome measure of DAS28-CRP CFB at Week 12 in a Phase 2 (a) study of peresolimab in participants with moderately-to-severely active RA.

FIG. 2 illustrates the significantly greater improvement in participants treated with 700 mg peresolimab (p<0.001), and 300 mg peresolimab (p<0.01) administered intravenously Q4W versus placebo in CDAI at Week 12 in a Phase 2 (a) study of peresolimab in participants with moderately-to-severely active RA.

FIG. 3 illustrates that patients treated with 700 mg and 300 mg peresolimab intravenously Q4W and achieving CDAI at week 14 maintained high level of CDAI responses through week 24 in a Phase 2 (a) study of peresolimab in participants with moderately-to-severely active RA. *p<0.05; ***p<0.001 vs. placebo; multiplicity is not controlled, nominal p-values are reported. ^{a}LSM change from baseline to Week 12, observed mean change from baseline from Weeks 14 to 24.

FIG. 4 illustrates that PD-1 mAb agonism correlates with PD-1 binding affinity, with higher affinity mAbs being more effective at inhibiting T cell proliferation. More specifically, human CFSE labelled PBMCs were incubated in the presence of SEB and 30 μg of one of the human IgG1 PD-1 mAbs A-D for three days. Proliferation upon mAb treatment was quantitated by assessing the number of CD4 T cells with reduced CFSE staining by using flow cytometry. The half-maximal inhibitory concentration (IC₅₀) for each PD-1 affinity variant is shown. Data are representative of two independent experiments.

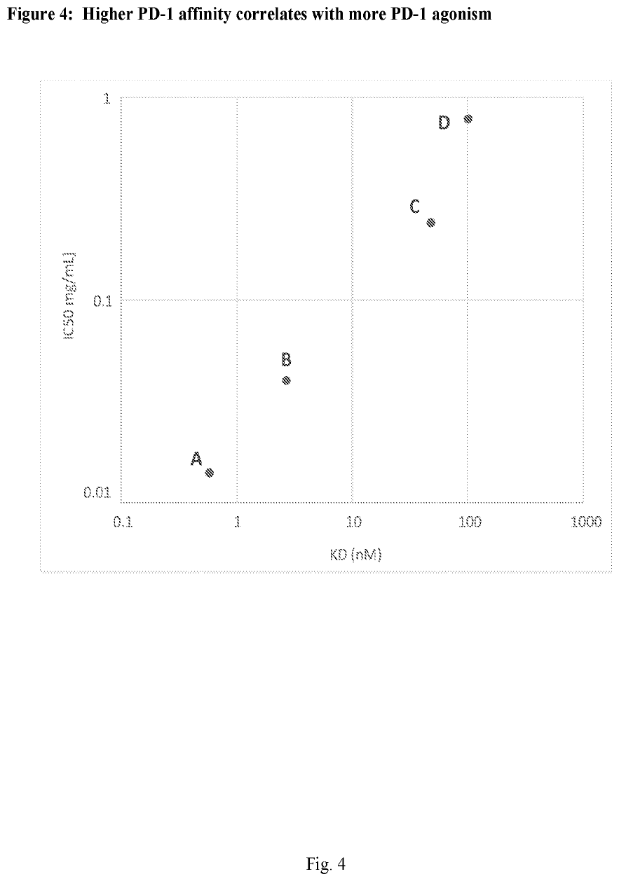

FIG. 5 illustrates the effect of PD-1 mAbs with human IgG1 Fc variants on SEB induced human PBMC proliferation. Human PBMCs were stimulated for three days in the presence of SEB and 30 g/mL of treatment with F1 derived PD-1 mAbs (A) or G2 derived PD-1 mAbs (B) in triplicates. Results shown are % T cell proliferation compared to untreated samples, mean+/−SEM from 6 donors for (A) and from 8-10 donors for (B).

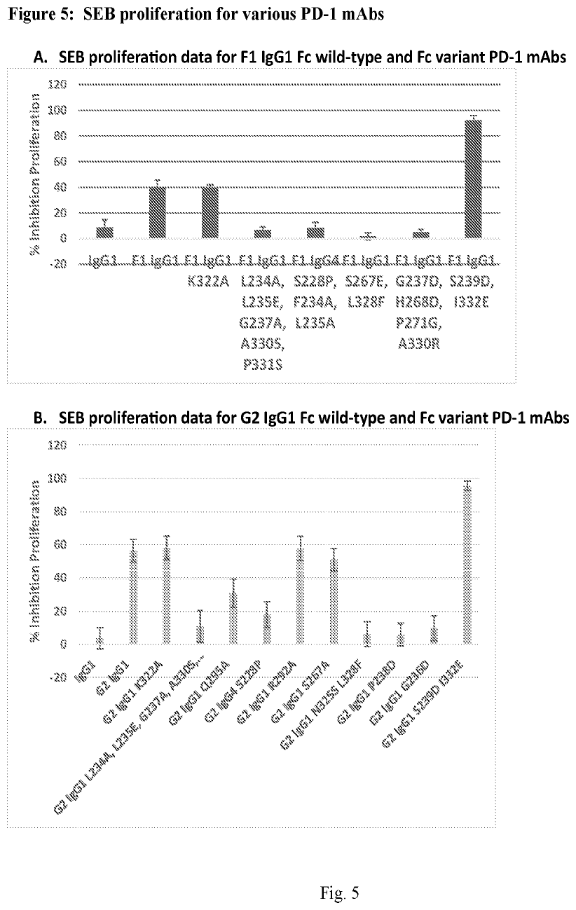

### DETAILED DESCRIPTION

The present disclosure describes novel PD-1 agonist antibodies as well as the drivers of PD-1 antibody mediated agonism and provides the first meaningful evidence of clinical efficacy for a PD-1 agonist in autoinflammatory and/or autoimmune disease and the first such result in rheumatology.

Unless defined otherwise, all technical and scientific terms used herein have the same meaning as commonly understood by one of skill in the art to which the disclosure pertains. Although any methods and materials similar to or equivalent to those described herein can be used in the practice or testing of the analogs, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “one.”

Certain abbreviations are defined as follows: ACR=American College of Rheumatology; ACR20=20% improvement in American College of Rheumatology criteria; ACR50=50% improvement in American College of Rheumatology criteria; ACR70=70% improvement in American College of Rheumatology criteria; AE=adverse event; CI=Confidence Interval; CFB=Change From Baseline; CDAI=Clinical Disease Activity Index; CRP=C-reactive protein; hsCRP=high sensitivity C-reactive protein; DAS28=Disease Activity Score modified to include the 28 diarthrodial joint count; HAQ-DI=Health Assessment Questionnaire-Disability Index; LSM=least squares mean; LDA=low disease activity; PRO=patient-reported outcome; SAE=serious adverse event; SDAI=Simplified Disease Activity Index; SF-36=Study 36 Item Short Form Health Survey; VAS=visual analog scale.

The term “about” when used to modify a numerically defined parameter means that the parameter may vary by as much as 10% above or below the stated numerical value for that parameter. For example, a dose of about 1000 mg administered subcutaneously once every 4 weeks (SC Q4W) should be understood to mean that the dose may vary between 900 mg SC Q4W and 1100 mg SC Q4W.

As used herein, the phrase “active rheumatoid arthritis” or “active RA” is used to mean RA with visible signs and symptoms (e.g., swelling, difficulty in flexion, etc.).

As used herein, an “antibody” is an immunoglobulin molecule capable of specific binding to a protein target, such as human PD-1, through at least one antigen recognition site, located in the variable region of the immunoglobulin molecule. The term “antibody” as used herein refers to an engineered, non-naturally occurring polypeptide complex, including an intact antibody and any antigen binding fragments thereof (i.e., “antigen binding portions” or “antibody binding domain” of an antibody). An intact antibody structurally comprises four polypeptide chains, two heavy chains and two light chains interconnected by disulfide bonds. Each heavy chain is comprised of an N-terminal heavy chain variable region (HCVR) and a heavy chain constant region. Each light chain is comprised of an N-terminal light chain variable region (LCVR) and a light chain constant region. HCVR and LCVR can be further subdivided into regions of high variability named as complementarity determining regions (CDRs) that are spaced by more conserved regions named as framework regions (FRs). Each HCVR and LCVR consists of three CDRs and four FRs arranged in the following order from the amino terminus to the carboxy terminus: FR1, CDR1, FR2, CDR2, FR3, CDR3, FR4. The variable regions of the heavy chain and light chain contain binding domains that interacts with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). A combination of IMGT and North CDR definitions were used for the exemplified human PD-1 agonist antibodies as described herein. It is further understood that the term “antibody” encompasses any cellular post-translational modifications to the antibody including, but not limited to, acylation and glycosylation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

As used herein, an “amino acid substitution” refers to the replacement of at least one existing amino acid residue in a given amino acid sequence with another different “replacement” amino acid residue.

As used herein, the term “variant Fc region” or “Fc region variant” refers to an amino acid sequence of a Fc region that differs from the sequence of a parent Fc region (or fragment thereof) by virtue of at least one amino acid substitution. Furthermore, substitutions are named herein by the amino acid in the parent Fc followed by the position number at which the substitution occurs followed by the amino acid substituted for the amino acid in the parent Fc region at the same position. For example, the human IgG1 Fc region variant P247I indicates that a proline residue at position 247 of the parent human IgG1 Fc region is substituted by an isoleucine residue).

As used herein, the term “disease-modifying anti-rheumatic drug (DMARD)” designates one drug of a class of drugs which is used in the treatment of autoinflammatory and/or autoimmune diseases to slow down disease progression. DMARDs include, csDMARDs, bDMARDs, and tsDMARDs.

As used herein, the term “conventional synthetic disease-modifying antirheumatic drug (csDMARD)” designates one drug of a class of synthetic drugs (not biologics) which are used in the treatment of autoinflammatory and/or autoimmune diseases. Exemplary csDMARDS include, but are not limited to, azathioprine, methotrexate, hydroxychloroquine, leflunomide, and sulfasalzine.

As used herein, the term “biologic disease-modifying antirheumatic drugs (bDMARDs)” designates drugs that have been produced using biotechnological methods. There are two main categories: TNF inhibitors, including, but not limited to, adalimumab, certolizumab pegol, etanercept, golimumab, and infliximab, and a biosimilar of any of the foregoing; and non-TNF inhibitors, including, but not limited to, tocilizumab, sarilumab, abatacept, anakinra, rituximab, and a biosimilar of any of the foregoing.

An “effective amount” of a therapeutic agent, e.g., a pharmaceutical formulation, refers to an amount effective, at dosages and for periods of time necessary, to achieve the desired therapeutic or prophylactic result. An “effective amount” of a therapeutic agent as provided herein can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In certain embodiments, the dosing is given by injections, e.g., intravenous or subcutaneous injections. In yet another embodiment, the therapeutic agent is administered using a syringe (e.g., prefilled or not) or an autoinjector.

A PD-1 polypeptide “extracellular domain” or “ECD” refers to a form of the PD-1 polypeptide that is essentially free of the transmembrane and cytoplasmic domains. Preferably, a PD-1 ECD has less than 1% of the transmembrane and cytoplasmic domain, more preferably, a PD-1 ECD has less than 0.5% of such domains. Even more preferably, human PD-1 ECD polypeptide is as shown in SEQ ID NO: 14, cynomolgus monkey PD-1 ECD polypeptide is as shown in SEQ ID NO: 16, and mouse PD-1 ECD is as shown in SEQ ID NO: 18. PD-1 polypeptide ECD may prepared using methods known in the art. Alternatively, human PD-1 polypeptide ECD may be purchased commercially from various vendors such as Sino Biological (Beijing, China; reference #10377-H08) and R&D Systems (Minneapolis, MN, USA; cat. #8986-PD). Cynomolgus PD-1 polypeptide ECD may be purchased commercially from R&D Systems (Minneapolis, MN, USA; cat. #8509-PD).

As used herein, “human PD-1 agonist antibody” refers to an antibody that binds to human PD-1, and, when administered in vivo, results in at least one significantly lessened autoimmune activity such as reduction in anti-double stranded DNA (ds-DNA) titers, inhibition of T cell activation, inhibition of T cell proliferation, reduction in disease scores or reduction in inflammatory cytokines.

The term “patient” or “subject” refers to any single subject for which therapy is desired or that is participating in a clinical trial, epidemiological study or used as a control. A “patient” or “subject” according to this disclosure includes, but is not limited to, an individual who may have had an inadequate response to, or who may have had failure to, or who may be intolerant to currently available DMARDs, csDMARDs, bDMARDs, or tsDMARDs, or who has not previously undergone treatment using bDMARDs.

As used herein, the term “peresolimab” refers to a monoclonal antibody that binds to human PD-1 and comprises two light chains and two heavy chains, and each of the light chains comprise the amino acid sequence of SEQ ID NO: 2 and each of the heavy chains comprise the amino acid sequence of SEQ ID NO: 1. Additionally, the light chain variable region and the heavy chain variable region of peresolimab comprise the amino acid sequence of SEQ ID NO: 4 and SEQ ID NO: 3, respectively. Furthermore, as used herein, the term “peresolimab” refers to the antibody known in the art as LY3462817 and as Antibody 1 in WO 2019/168745 and which is described in World Health Organization (2021). “International Nonproprietary Names for Pharmaceutical Substances (INN). Proposed INN: List 126” WHO Drug Information 35 (4), pages 1056-1057). The preparation of peresolimab was described in WO 2019/168745.

As used herein, the term “4D8” refers to a humanized rabbit monoclonal antibody that binds to human PD-1 as well as human PD-1 ECD. Monoclonal antibody 4D8 comprises two light chains and two heavy chains, and each of the light chains comprise the amino acid sequence of SEQ ID NO: 20 and each of the heavy chains comprise the amino acid sequence of SEQ ID NO: 19. Additionally, the light chain variable region and the heavy chain variable region of mAb 4D8 comprise the amino acid sequence of SEQ ID NO: 4 and SEQ ID NO: 3, respectively. As compared to peresolimab, the 4D8 mAb binds to a similar but not identical epitope on human PD-1 ECD and when bound to human PD-1 ECD, the 4D8 mAb, like peresolimab, does not block human PDL1 binding to human PD-1 ECD.

As used herein, the term “F1” refers to the antigen binding domain of mAb 4D8. As described herein the antigen binding domains, and variants thereof, of PD-1 antibodies such as peresolimab and mAb 4D8 can be paired with various human Fc regions such as, but not limited to, IgG1, IgG4 or Fc variants thereof for the purpose of, for example, assessing the impact of different human Fc regions on PD-1 antibody mediated agonism of the PD-1 pathway.

As used herein, the terms “G1” refers to an antigen binding domain closely related to the antigen binding domain of peresolimab. As described herein, G1 and variants thereof can be paired with various human Fc regions such as, but not limited to, IgG1, IgG4 or Fc variants thereof for the purpose of, for example, assessing the impact of different human Fc regions on PD-1 antibody mediated agonism of the PD-1 pathway.

As used herein, the term “G2” refers to an antigen binding domain identical in amino acid sequence to the antigen binding domain of peresolimab. As described herein, G2 and variants thereof can be paired with various human Fc regions such as, but not limited to, IgG1, IgG4 or Fc variants thereof for the purpose of, for example, assessing the impact of different human Fc regions on PD-1 antibody mediated agonism of the PD-1 pathway.

As used herein, the term “F2” refers to an antigen binding domain identical to F1 except F2 has two serine to cysteine amino acid substitutions in the HCVR of the antigen binding domain. More specifically, the HCVR region of F2 has cysteine residues at positions 36 and 51 rather than the serine residues at positions 36 and 51 in the HCVR of 4D8 as shown in SEQ ID NO: 21. As described herein, F2 and variants thereof can be paired with various human Fc regions such as, but not limited to, IgG1, IgG4 or Fc variants thereof for the purpose of, for example, assessing the impact of different human Fc regions on PD-1 antibody mediated agonism of the PD-1 pathway.

As used herein, the term “F series” and “G series” refers to antibodies or antigen binding fragments thereof which are humanized rabbit mAbs that bind to similar but not identical epitopes on human PD-1 ECD which do not block PDL 1 binding. The G series does not block PDL2 binding either, whereas the F series partially blocks PDL2.

As used herein, “pharmaceutically acceptable buffer” means any of the standard pharmaceutical buffers known to one of skill in the art.

As used herein, the term “targeted synthetic disease-modifying antirheumatic drugs (tsDMARDs)” designates drugs that are taken orally and target a specific molecular pathway, such as a Janus kinase inhibitor, e.g., tofacitinib, baricitinib, and upadacitinib.

As used herein, “treatment” or “treating” refers to all processes wherein there may be a slowing, controlling, or stopping of the progression of the diseases disclosed herein, but does not necessarily indicate a total elimination of all disease symptoms. Treatment includes administration of an antibody as described herein for treatment of a disease or condition in a patient, particularly in a human.

As used herein, the term an “inadequate response” or a “failure” to a previous treatment refers to: (1) a patient who has no meaningful clinical benefit (primary lack of efficacy); (2) a patient who has a measurable and meaningful response, but for whom response could be better, e.g., low disease activity or remission was not achieved; (3) a patient who, after an initial good response, worsens (secondary loss of efficacy); and (4) a patient who has a good response but discontinues because of a side effect (also termed “intolerance”). Patients who show TNF inadequate response (TNF-IR) or intolerance to TNF would be considered TNF failures. Patients who show methotrexate inadequate response (MTX-IR) or intolerance to MTX would be considered MTX failures. Patients who show DMARD inadequate response (DMARD-IR) or intolerance to DMARDs would be considered DMARD failures. In some embodiments of the disclosed methods, regimens, uses, and pharmaceutical compositions, the patient is a TNF failure, a MTX failure, or a DMARD failure.

As used herein, “clinical disease activity measures” of autoimmune disease include, but are not limited to, the American College of Rheumatology (ACR) 20, ACR50, ACR70; Disease Activity Score (DAS); Disease Activity Score-28 for RA with C-Reactive Protein (DAS28-CRP); Psoriasis Area and Severity Index (PASI) 50, PASI75, PASI90, PASI100; Systemic Lupus erythematosus disease activity index (SLEDAI); Mayo Score Disease activity index (DAI); Geboes score (GS); Robarts Histopathology index (RHI); Atopic dermatitis Severity Index (ADSI); hypoglycemic events, HbA1c, % time in range (blood glucose by CGM), total daily dose of insulin and/or the measurement of C-peptide under standardized conditions (including, but not limited to, total 4 hour C-peptide area under the curve (AUC) mixed-meal tolerance test (MMTT)) for Type I Diabetes; change from baseline in proteinuria and complete renal response, for example, for lupus nephritis; and EULAR Sjogren's syndrome disease activity index (ESSDAI)).

As used herein, the term “Clinical Disease Activity Index (CDAI)” refers to the well-known measure of disease activity in subjects having RA. CDAI is a composite score that integrates TJC28 (scored 0-28 with higher scores indicating higher disease activity), SJC28 (scored 0-28 with higher scores indicating higher disease activity), Patient's Global Assessment of Disease Activity (scored on a visual analogue scale from 0-10 cm with higher scores indicating higher disease activity), and Physician's Global Assessment of Disease Activity (scored on a visual analogue scale from 0-10 cm with higher scores indicating higher disease activity). The CDAI is calculated by summing the values of the 4 components. CDAI scores range from 0 to 76; lower scores indicated lower disease activity. A negative change from baseline indicates improvement in condition. CDAI may readily be assessed and calculated by one of ordinary skill in the art using the following formula:

$$CDAI = SJC ( 28 ) + T J C ( 28 ) + P G A + E G A$$

SJC (28): Swollen 28-Joint Count (shoulders, elbows, wrists, MCPs, PIPs including thumb IP, knees); TJC (28): Tender 28-Joint Count (shoulders, elbows, wrists, MCPs, PIPs including thumb IP, knees); PGA or PatGA: Patient Global disease Activity (patient's self-assessment of overall RA disease activity on a scale 1-10 where 10 is maximal activity); EGA: Evaluator's Global disease Activity (evaluator's assessment of overall RA disease activity on a scale 1-10 where 10 is maximal activity).

### Interpretation

| | |
|---|---|
| Remission | CDAI ≤ 2.8 |
| Low Disease Activity | CDAI > 2.8 and CDAI ≤ 10 |
| Moderate Disease Activity | CDAI >10 and CDAI ≤ 22 |
| High Disease Activity | CDAI > 22 |

As used herein, the term “36 Item Short Form Health Survey (SF-36)” refers to the well-known health-related survey that assesses participant's health status and consists of 36 questions covering 8 health domains: physical functioning, bodily pain, role limitations due to physical problems, role limitations due to emotional problems, general health, mental health, social functioning, and vitality. The 8 domains are combined to form 2 component scores mental (MCS) and physical (PCS). Each domain is scored by summing the individual items and transforming the scores into a 0 to 100 scale with higher scores indicating better health status.

As used herein, the term “Simplified Disease Activity Index (SDAI)” refers to the tool for measurement of disease activity in RA that integrates measures of physical examination, acute phase response, patient self-assessment, and evaluator assessment. The SDAI is calculated by adding together scores from 1) TJC28 (0 to 28), 2) SJC28 (0 to 28), 3) acute phase response using C-reactive protein (0.1 to 10.0 mg/dL), 4) Patient's Global Assessment of Disease Activity using VAS (0 to 10 cm), and 5) Physician's Global Assessment of Disease Activity using VAS (0 to 10 cm). Total Score scale range is 0 (remission) to 86 (high disease activity).

In one aspect of the present disclosure, there is provided a method of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., and that binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist.

In a second aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10.

In a third aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10,
   and the antibody binds at least one, at least two, at least three, or at least four of Fc [symbol] RI, Fc [symbol] RIIA_131H, FcY RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist.

In some embodiments, the HCVR of the antibody comprises the amino acid sequence of SEQ ID NO: 3, and the LCVR of the antibody comprises the amino acid sequence of SEQ ID NO: 4.

In some embodiments, the antibody comprises an immunoglobulin constant region.

In some embodiments, the immunoglobulin constant region is IgG1.

In some embodiments, the antibody comprises a heavy chain comprising the amino acid sequence of SEQ ID NO: 1 and a light chain comprising the amino acid sequence of SEQ ID NO: 2.

In some embodiments, the antibody comprises two heavy chains and two light chains, wherein each heavy chain comprises the amino acid sequence of SEQ ID NO: 1, and each light chain comprises the amino acid sequence of SEQ ID NO: 2.

In some embodiments, the antibody is peresolimab.

In some embodiments, the autoinflammatory and/or autoimmune disease is RA, SLE, lupus nephritis (LN), cutaneous lupus erythematosus (CLE), giant cell arteritis (GCA), vasculitis, ulcerative colitis (UC), Crohn's disease (CD), Sjogren's syndrome (SjS), or T1DM.

In another aspect, the antibody is administered subcutaneously at about 75 mg to about 1200 mg once every week (Q1W) to the patient.

In another aspect, the antibody is administered subcutaneously at about 100 mg to about 1150 mg Q1W to the patient.

In another aspect, the antibody is administered subcutaneously at about 150 mg to about 1100 mg Q1W to the patient.

In another aspect, the antibody is administered subcutaneously at about 200 mg to about 1050 mg Q1W to the patient.

In another aspect, the antibody is administered subcutaneously at about 250 mg to about 1000 mg Q1W to the patient.

In another aspect, the antibody is administered subcutaneously at about 300 mg to about 950 mg Q1W to the patient.

In another aspect, the antibody is administered subcutaneously at about 350 mg to about 900 mg Q1W to the patient.

In another aspect, the antibody is administered subcutaneously at about 400 mg to about 850 mg Q1W to the patient.

In another aspect, the antibody is administered subcutaneously at about 450 mg to about 800 mg Q1W to the patient.

In another aspect, the antibody is administered subcutaneously at about 500 mg to about 750 mg Q1W to the patient.

In another aspect, the antibody is administered subcutaneously at about 550 mg to about 700 mg Q1W to the patient.

In another aspect, the antibody is administered subcutaneously at about 600 mg to about 700 mg Q1W to the patient.

In another aspect, the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q1W to the patient.

In another aspect, the antibody is administered subcutaneously at about 75 mg to about 1200 mg once every four weeks (Q4W) to the patient.

In another aspect, the antibody is administered subcutaneously at about 100 mg to about 1150 mg Q4W to the patient.

In another aspect, the antibody is administered subcutaneously at about 150 mg to about 1100 mg Q4W to the patient.

In another aspect, the antibody is administered subcutaneously at about 200 mg to about 1050 mg Q4W to the patient.

In another aspect, the antibody is administered subcutaneously at about 250 mg to about 1000 mg Q4W to the patient.

In another aspect, the antibody is administered subcutaneously at about 300 mg to about 950 mg Q4W to the patient.

In another aspect, the antibody is administered subcutaneously at about 350 mg to about 900 mg Q4W to the patient.

In another aspect, the antibody is administered subcutaneously at about 400 mg to about 850 mg Q4W to the patient.

In another aspect, the antibody is administered subcutaneously at about 450 mg to about 800 mg Q4W to the patient.

In another aspect, the antibody is administered subcutaneously at about 500 mg to about 750 mg Q4W to the patient.

In another aspect, the antibody is administered subcutaneously at about 550 mg to about 700 mg Q4W to the patient.

In another aspect, the antibody is administered subcutaneously at about 600 mg to about 700 mg Q4W to the patient.

In another aspect, the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient.

In other embodiments, the patient is one who has had an inadequate response to, who has had failure to, or who has been intolerant to standard treatments for an autoinflammatory and/or autoimmune disease.

In other embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In other embodiments, the patient is one who has not previously undergone treatment using a bDMARD.

In other embodiments, the patient is one who has had an inadequate response to or who has had failure to: 1) less than three bDMARDs, 2) less than three tsDMARDs, or 3) less than three of any combination of inadequate responses or failures to bDMARDs and tsDMARDs. For clarity, in such embodiments, the patient may be one who has had an inadequate response to one bDMARD and one tsDMARD or may be one who has had a failure to one bDMARD and an inadequate response to one tsDMARD whereas the patient would not be one who has had two inadequate responses to two different bDMARDs and a failure to one tsDMARD, for example.

In other embodiments, the patient is an adult patient with moderately-to-severely active RA, wherein the antibody is administered subcutaneously at about 75 mg to about 1200 mg Q4W to the patient.

In other embodiments, the patient is an adult patient with moderately-to-severely active RA, wherein the antibody is administered subcutaneously at about 100 mg to about 1150 mg Q4W to the patient.

In other embodiments, the patient is an adult patient with moderately-to-severely active RA, wherein the antibody is administered subcutaneously at about 150 mg to about 1100 mg Q4W to the patient.

In other embodiments, the patient is an adult patient with moderately-to-severely active RA, wherein the antibody is administered subcutaneously at about 200 mg to about 1050 mg Q4W to the patient.

In other embodiments, the patient is an adult patient with moderately-to-severely active RA, wherein the antibody is administered subcutaneously at about 250 mg to about 1000 mg Q4W to the patient.

In other embodiments, the patient is an adult patient with moderately-to-severely active RA, wherein the antibody is administered subcutaneously at about 300 mg to about 950 mg Q4W to the patient.

In other embodiments, the patient is an adult patient with moderately-to-severely active RA, wherein the antibody is administered subcutaneously at about 350 mg to about 900 mg Q4W to the patient.

In other embodiments, the patient is an adult patient with moderately-to-severely active RA, wherein the antibody is administered subcutaneously at about 400 mg to about 850 mg Q4W to the patient.

In other embodiments, the patient is an adult patient with moderately-to-severely active RA, wherein the antibody is administered subcutaneously at about 450 mg to about 800 mg Q4W to the patient.

In other embodiments, the patient is an adult patient with moderately-to-severely active RA, wherein the antibody is administered subcutaneously at about 500 mg to about 750 mg Q4W to the patient.

In other embodiments, the patient is an adult patient with moderately-to-severely active RA, wherein the antibody is administered subcutaneously at about 550 mg to about 700 mg Q4W to the patient.

In other embodiments, the patient is an adult patient with moderately-to-severely active RA, wherein the antibody is administered subcutaneously at about 600 mg to about 700 mg Q4W to the patient.

In other embodiments, the patient is an adult patient with moderately-to-severely active RA, wherein the antibody is administered subcutaneously about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient.

In another embodiment, if the patient achieves clinical responses with the clinical disease activity index (CDAI)≤10, the antibody is administered subcutaneously once every twelve weeks (Q12W) to the patient, or the antibody is continued to be administered subcutaneously Q4W to the patient.

In other embodiments, the patient is an adult patient with moderately-to-severely active RA, wherein the antibody is administered subcutaneously at about 75 mg to about 1200 mg Q12W to the patient.

In other embodiments, the patient is an adult patient with moderately-to-severely active RA, wherein the antibody is administered subcutaneously at about 100 mg to about 1150 mg Q12W to the patient.

In other embodiments, the patient is an adult patient with moderately-to-severely active RA, wherein the antibody is administered subcutaneously at about 150 mg to about 1100 mg Q12W to the patient.

In other embodiments, the patient is an adult patient with moderately-to-severely active RA, wherein the antibody is administered subcutaneously at about 200 mg to about 1050 mg Q12W to the patient.

In other embodiments, the patient is an adult patient with moderately-to-severely active RA, wherein the antibody is administered subcutaneously at about 250 mg to about 1000 mg Q12W to the patient.

In other embodiments, the patient is an adult patient with moderately-to-severely active RA, wherein the antibody is administered subcutaneously at about 300 mg to about 950 mg Q12W to the patient.

In other embodiments, the patient is an adult patient with moderately-to-severely active RA, wherein the antibody is administered subcutaneously at about 350 mg to about 900 mg Q12W to the patient.

In other embodiments, the patient is an adult patient with moderately-to-severely active RA, wherein the antibody is administered subcutaneously at about 400 mg to about 850 mg Q12W to the patient.

In other embodiments, the patient is an adult patient with moderately-to-severely active RA, wherein the antibody is administered subcutaneously at about 450 mg to about 800 mg Q12W to the patient.

In other embodiments, the patient is an adult patient with moderately-to-severely active RA, wherein the antibody is administered subcutaneously at about 500 mg to about 750 mg Q12W to the patient.

In other embodiments, the patient is an adult patient with moderately-to-severely active RA, wherein the antibody is administered subcutaneously at about 550 mg to about 700 mg Q12W to the patient.

In other embodiments, the patient is an adult patient with moderately-to-severely active RA, wherein the antibody is administered subcutaneously at about 600 mg to about 700 mg Q12W to the patient.

In other embodiments, the patient is an adult patient with moderately-to-severely active RA, wherein the antibody is administered subcutaneously about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient.

In some embodiments, the antibody is peresolimab.

In some embodiments, patients administered peresolimab at a dose, frequency and route disclosed above results in a statistically significant increase (as compared to patients administered a placebo at the same dose, frequency and route) in measures of disease activity such as:
proportion of patients achieving ACR20;
proportion of patients achieving ACR50;
proportion of patients achieving ACR70;
proportion of patients achieving LDA or remission for DAS28-CRP or DAS28-hsCRP, DAS28-ESR, SDAI, and CDAI;
change from baseline for mean DAS28-CRP or DAS28-hsCRP, SDAI, and CDAI; and/or change from baseline for ACR core set values of 68 tender joint count, 66 swollen joint count, and Physician's Global Assessment of Disease Activity (VAS), at Week 12, Week 24, Week 48, and/or Week 60.

In some embodiments, patients administered peresolimab at a dose, frequency and route disclosed above results in a statistically significant increase (as compared to patients administered a placebo at the same dose, frequency and route) in patient reported outcome measures such as:
change from baseline for patient-reported ACR core set values such as Patient's Global Assessment of Disease Activity (VAS), Patient's Assessment of Arthritis Pain (VAS), and/or patient's assessment of physical function using HAQ-DI;
change from baseline for the duration and severity of morning joint stiffness; and/or Functional Assessment of Chronic Illness Therapy-Fatigue (FACIT F) scores, at Week 12, Week 24, Week 48, and/or Week 60;
change from baseline for SF-36 domains, SF-36 Physical Component Summary, and/or SF-36 Mental Component Summary at Week 12, Week 24, Week 48, and/or Week 60.

Also provided herein, is an antibody that binds human PD-1 for use in the treatment of an autoinflammatory and/or autoimmune disease, comprising administering about 75 mg to about 1200 mg of the antibody to a patient in need thereof, wherein the antibody comprises a HCVR and a LCVR, wherein the HCVR comprises HCDR1, HCDR2, and HCDR3, and the LCVR comprises LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10.

In some embodiments, the antibody for use in the treatment of an autoinflammatory and/or autoimmune disease comprises a HCVR comprising the amino acid sequence of SEQ ID NO: 3, and a LCVR comprising the amino acid sequence of SEQ ID NO: 4.

In some embodiments, the antibody for use in the treatment of the autoinflammatory and/or autoimmune disease comprises an immunoglobulin constant region.

In some embodiments, the antibody for use in the treatment of the autoinflammatory and/or autoimmune disease comprises an immunoglobulin constant region, wherein the immunoglobulin constant region is IgG1.

In some embodiments, the antibody for use in the treatment of the autoinflammatory and/or autoimmune disease comprises a heavy chain comprising the amino acid sequence of SEQ ID NO: 1 and a light chain comprising the amino acid sequence of SEQ ID NO: 2.

In some embodiments, the antibody for use in the treatment of the autoinflammatory and/or autoimmune disease comprises two heavy chains and two light chains, wherein each heavy chain comprises the amino acid sequence of SEQ ID NO: 1, and each light chain comprises the amino acid sequence of SEQ ID NO: 2.

In some embodiments, the antibody for use in the treatment of the autoinflammatory and/or autoimmune disease is peresolimab.

In some embodiments, the antibody for use in the treatment of the autoinflammatory and/or autoimmune disease is administered subcutaneously at about 75 mg to about 1200 mg Q1W to the patient.

In some embodiments, the antibody for use in the treatment of the autoinflammatory and/or autoimmune disease is administered subcutaneously at about 100 mg to about 1150 mg Q1W to the patient.

In some embodiments, the antibody for use in the treatment of the autoinflammatory and/or autoimmune disease is administered subcutaneously at about 150 mg to about 1100 mg Q1W to the patient.

In some embodiments, the antibody for use in the treatment of the autoinflammatory and/or autoimmune disease is administered subcutaneously at about 200 mg to about 1050 mg Q1W to the patient.

In some embodiments, the antibody for use in the treatment of the autoinflammatory and/or autoimmune disease is administered subcutaneously at about 250 mg to about 1000 mg Q1W to the patient.

In some embodiments, the antibody for use in the treatment of the autoinflammatory and/or autoimmune disease is administered subcutaneously at about 300 mg to about 950 mg Q1W to the patient.

In some embodiments, the antibody for use in the treatment of the autoinflammatory and/or autoimmune disease is administered subcutaneously at about 350 mg to about 900 mg Q1W to the patient.

In some embodiments, the antibody for use in the treatment of the autoinflammatory and/or autoimmune disease is administered subcutaneously at about 400 mg to about 850 mg Q1W to the patient.

In some embodiments, the antibody for use in the treatment of the autoinflammatory and/or autoimmune disease is administered subcutaneously at about 450 mg to about 800 mg Q1W to the patient.

In some embodiments, the antibody for use in the treatment of the autoinflammatory and/or autoimmune disease is administered subcutaneously at about 500 mg to about 750 mg Q1W to the patient.

In some embodiments, the antibody for use in the treatment of the autoinflammatory and/or autoimmune disease is administered subcutaneously at about 550 mg to about 700 mg Q1W to the patient.

In some embodiments, the antibody for use in the treatment of the autoinflammatory and/or autoimmune disease is administered subcutaneously at about 600 mg to about 700 mg Q1W to the patient.

In some embodiments, the antibody for use in the treatment of the autoinflammatory and/or autoimmune disease is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q1W to the patient.

In some embodiments, the antibody for use in the treatment of the autoinflammatory and/or autoimmune disease is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient.

In some embodiments, the antibody for use in the treatment of the autoinflammatory and/or autoimmune disease is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient.

In some embodiments, the antibody is for use in the treatment of an autoinflammatory and/or autoimmune disease, wherein the autoinflammatory and/or autoimmune disease is RA, SLE, LN, CLE, GCA, vasculitis, UC, CD, SjS, or T1DM.

In some embodiments, the antibody is for use in the treatment of an autoinflammatory and/or autoimmune disease, wherein the patient has had an inadequate response to, has had a failure to, or who has been intolerant to one or more csDMARD, bDMARD, tsDMARD, TNF inhibitor, and/or non-TNF inhibitor.

In some embodiments, the antibody is for use in the treatment of an autoinflammatory and/or autoimmune disease, wherein the patient has not previously undergone treatment using a bDMARD.

In some embodiments, the antibody is for use in the treatment of an autoinflammatory and/or autoimmune disease, wherein the patient is an adult patient with moderately-to-severely active RA, and wherein the antibody is administered subcutaneously about 75 mg to about 1200 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 100 mg to about 1150 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 150 mg to about 1100 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 200 mg to about 1050 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 250 mg to about 1000 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 300 mg to about 950 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 350 mg to about 900 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 400 mg to about 850 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 450 mg to about 800 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 500 mg to about 750 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 550 mg to about 700 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 600 mg to about 700 mg Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient.

In some embodiments, the antibody is for use in the treatment of an autoinflammatory and/or autoimmune disease, wherein if the patient achieves clinical responses with the CDAI ≤10, the antibody is administered subcutaneously Q12W to the patient, or the antibody is continued to be administered subcutaneously Q4W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 75 mg to about 1200 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 100 mg to about 1150 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 150 mg to about 1100 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 200 mg to about 1050 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 250 mg to about 1000 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 300 mg to about 950 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 350 mg to about 900 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 400 mg to about 850 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 450 mg to about 800 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 500 mg to about 750 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 550 mg to about 700 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 600 mg to about 700 mg Q12W to the patient.

In some embodiments, the antibody is for use in the treatment of an autoinflammatory and/or autoimmune disease, wherein the patient is an adult patient with moderately-to-severely active RA, and wherein the antibody is administered subcutaneously about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient.

In some embodiments, the antibody is for use in the treatment of an autoinflammatory and/or autoimmune disease wherein patients administered the PD-1 agonist antibody at a dose, frequency and route disclosed herein results in a statistically significant increase (as compared to patients administered a placebo at the same dose, frequency and route) in measures of disease activity such as:
proportion of patients achieving ACR20;
proportion of patients achieving ACR50;
proportion of patients achieving ACR70;
proportion of patients achieving LDA or remission for DAS28-CRP or DAS28-hsCRP, DAS28-ESR, SDAI, and CDAI;
change from baseline for mean DAS28-CRP or DAS28-hsCRP, SDAI, and CDAI; and/or change from baseline for ACR core set values of 68 tender joint count, 66 swollen joint count, and Physician's Global Assessment of Disease Activity (VAS), at Week 12, Week 24, Week 48, and/or Week 60.

In some embodiments, the antibody is for use in the treatment of an autoinflammatory and/or autoimmune disease wherein patients administered peresolimab at a dose, frequency and route disclosed above results in a statistically significant increase (as compared to patients administered a placebo at the same dose, frequency and route) in patient reported outcome measures such as:
change from baseline for patient-reported ACR core set values such as Patient's Global Assessment of Disease Activity (VAS), Patient's Assessment of Arthritis Pain (VAS), and/or patient's assessment of physical function using HAQ-DI;
change from baseline for the duration and severity of morning joint stiffness; and/or Functional Assessment of Chronic Illness Therapy-Fatigue (FACIT F) scores, at Week 12, Week 24, Week 48, and/or Week 60;
change from baseline for SF-36 domains, SF-36 Physical Component Summary, and/or SF-36 Mental Component Summary at Week 12, Week 24, Week 48, and/or Week 60.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6.0 pM to about 1 μM to Fc [symbol] RI;
(ii) about 20 nM to about 10 μM to Fc [symbol] RIIA_131H;
(iii) about 4 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6.0 pM to about 1 μM to Fc [symbol] RI;
(ii) about 20 nM to about 10 μM to Fc [symbol] RIIA_131H;
(iii) about 4 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 nM to Fc [symbol] RI;
(ii) about 30 nM to about 10 μM to Fc [symbol] RIIA_131H;
(iii) about 5 nM to about 5 μM to Fc [symbol] RIIA_131R;
(iv) about 20 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc RIIA_131R, Fc [symbol] RIIb, Fc [symbol] RIIIA_158F and Fc [symbol] RIIIA_158V with an affinity of:
(i) about 7.5 pM to about 100 nM to Fc [symbol] RI;
(ii) about 30 nM to about 10 μM to Fc [symbol] RIIA_131H;
(iii) about 5 nM to about 5 μM to Fc [symbol] RIIA_131R;
(iv) about 20 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg once every 12 weeks Q12W to the patient. In some embodiments, the patient is one who has had an inadequate response to, who has had failure to, or who has been intolerant to one or more of the following drugs:
a csDMARD,
a bDMARD,
a tsDMARD,
one or more TNF inhibitors, or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 pM to Fc [symbol] RI;
(ii) about 50 nM to about 1 μM to Fc [symbol] RIIA_131H;
(iii) about 5 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 pM to Fc [symbol] RI;
(ii) about 50 nM to about 1 μM to Fc [symbol] RIIA_131H;
(iii) about 5 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6 pM to about 100 pM to Fc [symbol] RI;
(ii) about 50 nM to about 250 nM to Fc [symbol] RIIA_131H;
(iii) about 50 nM to about 250 nM to Fc [symbol] RIIA_131R;
(iv) about 20 nM to about 250 nM to Fc [symbol] RIIb;
(v) about 10 nM to about 250 nM to Fc [symbol] RIIIA_158V; and
(vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6 pM to about 100 pM to Fc [symbol] RI;
(ii) about 50 nM to about 250 nM to Fc [symbol] RIIA_131H;
(iii) about 50 nM to about 250 nM to Fc [symbol] RIIA_131R;
(iv) about 20 nM to about 250 nM to Fc [symbol] RIIb;
(v) about 10 nM to about 250 nM μM to Fc [symbol] RIIIA_158V; and
(vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 50 pM to about 100 pM to Fc [symbol] RI;
(ii) about 250 nM to about 1 μM to Fc [symbol] RIIA_131H;
(iii) about 50 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA 131R, Fc [symbol] RIIb, Fc [symbol] RIIIA_158F and Fc [symbol] RIIIA_158V with an affinity of:
(i) about 50 pM to about 100 pM to Fc [symbol] RI;
(ii) about 250 nM to about 1 μM to Fc [symbol] RIIA_131H;
(iii) about 50 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 100 pM to Fc [symbol] RI;
(ii) about 900 pM to Fc [symbol] RIIA_131H;
(iii) about 1.5 μM to Fc [symbol] RIIA_131R;
(iv) about 5.2 μM to Fc [symbol] RIIb;
(v) about 1.5 μM to Fc [symbol] RIIIA_158V; and
(vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA 131R, Fc [symbol] RIIb, Fc [symbol] RIIIA_158F and Fc [symbol] RIIIA_158V with an affinity of:
(i) about 100 pM to Fc [symbol] RI;
(ii) about 900 pM to Fc [symbol] RIIA_131H;
(iii) about 1.5 μM to Fc [symbol] RIIA_131R;
(iv) about 5.2 μM to Fc [symbol] RIIb;
(v) about 1.5 μM to Fc [symbol] RIIIA_158V; and
(vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10.

In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments the antibody is not peresolimab. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein the HCDR1 comprises the amino acid sequence of SEQ ID NO: 5, the HCDR2 comprises the amino acid sequence of SEQ ID NO: 6, the HCDR3 comprises the amino acid sequence of SEQ ID NO: 7, the LCDR1 comprises the amino acid sequence of SEQ ID NO: 8, the LCDR2 comprises the amino acid sequence of SEQ ID NO: 9, and the LCDR3 comprises the amino acid sequence of SEQ ID NO: 10.

In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments the antibody is not peresolimab. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein the HCDR1 comprises the amino acid sequence of SEQ ID NO: 5, the HCDR2 comprises the amino acid sequence of SEQ ID NO: 6, the HCDR3 comprises the amino acid sequence of SEQ ID NO: 7, the LCDR1 comprises the amino acid sequence of SEQ ID NO: 8, the LCDR2 comprises the amino acid sequence of SEQ ID NO: 9, and the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
(i) about 6.0 pM to about 1 μM to Fc [symbol] RI;
(ii) about 20 nM to about 10 μM to Fc [symbol] RIIA_131H;
(iii) about 4 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 6.0 pM to about 1 μM to Fc [symbol] RI;
   (ii) about 20 nM to about 10 μM to Fc [symbol] RIIA_131H;
   (iii) about 4 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 7.5 pM to about 100 nM to Fc [symbol] RI;
   (ii) about 30 nM to about 10 μM to Fc [symbol] RIIA_131H;
   (iii) about 5 nM to about 5 μM to Fc [symbol] RIIA_131R;
   (iv) about 20 nM to about 10 μM to Fc [symbol] RIIb;
   (v) about 100 pM to about 10 M to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 7.5 pM to about 100 nM to Fc [symbol] RI;
   (ii) about 30 nM to about 10 μM to Fc [symbol] RIIA_131H;
   (iii) about 5 nM to about 5 μM to Fc [symbol] RIIA_131R;
   (iv) about 20 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 7.5 pM to about 100 pM to Fc [symbol] RI;
   (ii) about 50 nM to about 1 μM to Fc [symbol] RIIA_131H;
   (iii) about 5 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 10 nM to about 1 μM to Fc [symbol] RIIIA 158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 7.5 pM to about 100 pM to Fc [symbol] RI;
   (ii) about 50 nM to about 1 μM to Fc [symbol] RIIA_131H;
   (iii) about 5 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 6 pM to about 100 pM to Fc [symbol] RI;
   (ii) about 50 nM pM to about 250 nM to Fc [symbol] RIIA_131H;
   (iii) about 50 nM to about 250 nM to Fc [symbol] RIIA_131R;
   (iv) about 20 nM to about 250 nM to Fc [symbol] RIIb;
   (v) about 10 nM to about 250 nM to Fc [symbol] RIIIA_158V; and
   (vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 6 pM to about 100 pM to Fc [symbol] RI;
   (ii) about 50 nM pM to about 250 nM to Fc [symbol] RIIA_131H;
   (iii) about 50 nM to about 250 nM to Fc [symbol] RIIA_131R;
   (iv) about 20 nM to about 250 nM to Fc [symbol] RIIb;
   (v) about 10 nM to about 250 nM to Fc [symbol] RIIIA_158V; and
   (vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 50 pM to about 100 pM to Fc [symbol] RI;
   (ii) about 250 nM to about 1 μM to Fc [symbol] RIIA_131H;
   (iii) about 50 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 50 pM to about 100 pM to Fc [symbol] RI;
   (ii) about 250 nM to about 1 μM to Fc [symbol] RIIA_131H;
   (iii) about 50 nM to about 1 μM to Fc [symbol] RIIA_131R;
   (iv) about 20 nM to about 1 μM to Fc [symbol] RIIb;
   (v) about 10 nM to about 1 M to Fc [symbol] RIIIA_158V; and
   (vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 100 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 1.5 μM to Fc [symbol] RIIIA_158V; and
   (vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 100 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 1.5 μM to Fc [symbol] RIIIA_158V; and
   (vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 55 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 340 nM to Fc [symbol] RIIIA_158V; and
   (vi) about 1.7 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q4W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 55 PM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 340 nM to Fc [symbol] RIIIA_158V; and
   (vi) about 1.7 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q12W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD.

In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, FcY RIIA_131R, Fc [symbol] RIIb, Fc [symbol] RIIIA_158F and Fc [symbol] RIIIA_158V with an affinity of:
(i) about 6.0 pM to about 1 μM to Fc [symbol] RI;
(ii) about 20 nM to about 10 μM to Fc [symbol] RIIA_131H;
(iii) about 4 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6.0 pM to about 1 μM to Fc [symbol] RI;
(ii) about 20 nM to about 10 μM to Fc [symbol] RIIA_131H;
(iii) about 4 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 nM to Fc [symbol] RI;
(ii) about 30 nM to about 10 μM to Fc [symbol] RIIA_131H;
(iii) about 5 nM to about 5 μM to Fc [symbol] RIIA_131R;
(iv) about 20 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 nM to Fc [symbol] RI;
(ii) about 30 nM to about 10 μM to Fc [symbol] RIIA_131H;
(iii) about 5 nM to about 5 μM to Fc [symbol] RIIA_131R;
(iv) about 20 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD,
   a bDMARD,
   a tsDMARD,
   one or more TNF inhibitors, or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 pM to Fc [symbol] RI;
(ii) about 50 nM to about 1 μM to Fc [symbol] RIIA_131H;
(iii) about 5 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 pM to Fc [symbol] RI;
(ii) about 50 nM to about 1 μM to Fc [symbol] RIIA_131H;
(iii) about 5 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA 131R, Fc [symbol] RIIb, Fc [symbol] RIIIA_158F and Fc [symbol] RIIIA_158V with an affinity of:
(i) about 6 pM to about 100 pM to Fc [symbol] RI;
(ii) about 50 nM to about 250 nM to Fc [symbol] RIIA_131H;
(iii) about 50 nM to about 250 nM to Fc [symbol] RIIA_131R;
(iv) about 20 nM to about 250 nM to Fc [symbol] RIIb;
(v) about 10 nM to about 250 nM to Fc [symbol] RIIIA_158V; and
(vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6 pM to about 100 pM to Fc [symbol] RI;
(ii) about 50 nM to about 250 nM to Fc [symbol] RIIA_131H;
(iii) about 50 nM to about 250 nM to Fc [symbol] RIIA_131R;
(iv) about 20 nM to about 250 nM to Fc [symbol] RIIb;
(v) about 10 nM to about 250 nM μM to Fc [symbol] RIIIA_158V; and
(vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 50 pM to about 100 pM to Fc [symbol] RI;
(ii) about 250 nM to about 1 μM to Fc [symbol] RIIA_131H;
(iii) about 50 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 50 pM to about 100 pM to Fc [symbol] RI;
(ii) about 250 nM to about 1 μM to Fc [symbol] RIIA_131H;
(iii) about 50 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 100 pM to Fc [symbol] RI;
(ii) about 900 pM to Fc [symbol] RIIA_131H;
(iii) about 1.5 μM to Fc [symbol] RIIA_131R;
(iv) about 5.2 μM to Fc [symbol] RIIb;
(v) about 1.5 μM to Fc [symbol] RIIIA_158V; and
(vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 100 pM to Fc [symbol] RI;
(ii) about 900 pM to Fc [symbol] RIIA_131H;
(iii) about 1.5 μM to Fc [symbol] RIIA_13IR;
(iv) about 5.2 μM to Fc [symbol] RIIb;
(v) about 1.5 μM to Fc [symbol] RIIIA_158V; and
(vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10.

In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments the antibody is not peresolimab. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments the antibody is not peresolimab. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 1 pM to about 1 μM to Fc [symbol] RI;
   (ii) about 10 nM to about 10 μM to Fc [symbol] RIIA_131H;
   (iii) about 1 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 1 pM to about 1 μM to Fc [symbol] RI;
   (ii) about 10 nM to about 10 μM to Fc [symbol] RIIA_131H;
   (iii) about 1 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 6.0 pM to about 1 μM to Fc [symbol] RI;
   (ii) about 20 nM to about 10 μM to Fc [symbol] RIIA_131H;
   (iii) about 4 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc RIIA_131H, Fc [symbol] RIIA_131R, Fc [symbol] RIIb, Fc [symbol] RIIIA_158F and Fc [symbol] RIIIA_158V with an affinity of
   (i) about 6.0 pM to about 1 μM to Fc [symbol] RI;
   (ii) about 20 nM to about 10 μM to Fc [symbol] RIIA_131H;
   (iii) about 4 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 7.5 pM to about 100 nM to Fc [symbol] RI;
   (ii) about 30 nM to about 10 μM to Fc [symbol] RIIA_131H;
   (iii) about 5 nM to about 5 μM to Fc [symbol] RIIA_131R;
   (iv) about 20 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein

the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 7.5 pM to about 100 nM to Fc [symbol] RI;
   (ii) about 30 nM to about 10 μM to Fc [symbol] RIIA_131H;
   (iii) about 5 nM to about 5 μM to Fc [symbol] RIIA_131R;
   (iv) about 20 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 7.5 pM to about 100 pM to Fc [symbol] RI;
   (ii) about 50 nM to about 1 μM to Fc [symbol] RIIA_131H;
   (iii) about 5 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 10 nM to about 1 μM to Fc [symbol] RIIIA 158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 7.5 pM to about 100 pM to Fc [symbol] RI;
   (ii) about 50 nM to about 1 μM to Fc [symbol] RIIA_131H;
   (iii) about 5 nM to about 1 μM to Fc [symbol] RIIA_131R;
   (iv) about 20 nM to about 1 μM to Fc [symbol] RIIb;
   (v) about 10 nM to about 1 μM to Fc RIIIA_158V; and
   (vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 6 pM to about 100 pM to Fc [symbol] RI;
   (ii) about 50 nM pM to about 250 nM to Fc [symbol] RIIA_131H;
   (iii) about 50 nM to about 250 nM to Fc [symbol] RIIA_131R;
   (iv) about 20 nM to about 250 nM to Fc [symbol] RIIb;
   (v) about 10 nM to about 250 nM to Fc [symbol] RIIIA_158V; and
   (vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 6 pM to about 100 pM to Fc [symbol] RI;
   (ii) about 50 nM pM to about 250 nM to Fc [symbol] RIIA_131H;
   (iii) about 50 nM to about 250 nM to Fc [symbol] RIIA_131R;
   (iv) about 20 nM to about 250 nM to Fc [symbol] RIIb;
   (v) about 10 nM to about 250 nM to Fc [symbol] RIIIA_158V; and
   (vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 50 pM to about 100 pM to Fc [symbol] RI;
   (ii) about 250 nM to about 1 μM to Fc [symbol] RIIA_131H;
   (iii) about 50 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 50 pM to about 100 pM to Fc [symbol] RI;
   (ii) about 250 nM to about 1 μM to Fc [symbol] RIIA_131H;
   (iii) about 50 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 100 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 1.5 μM to Fc [symbol] RIIIA_158V; and
   (vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 100 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 1.5 μM to Fc [symbol] RIIIA_158V; and
   (vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RITIA_158F and Fc [symbol] RIIIA_158V with an affinity of:
   (i) about 55 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 340 nM to Fc [symbol] RIIIA_158V; and
   (vi) about 1.7 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q6W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 55 PM to Fc [symbol] RI;
   (ii) about 900 pM to Fc [symbol] RIIA_131H;
   (iii) about 1.5 M to Fc [symbol] RIIA_131R;
   (iv) about 5.2 μM to Fc [symbol] RIIb;
   (v) about 340 nM to Fc [symbol] RIIIA_158V; and
   (vi) about 1.7 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q8W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect of the present disclosure, there is provided a method of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and the LCDR3 comprises the amino acid sequence of SEQ ID NO: 10, wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 6.0 pM to about 1 μM to Fc [symbol] RI;
(ii) about 20 nM to about 10 μM to Fc [symbol] RIIA_131H;
(iii) about 4 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has
   been intolerant to one or more of the following drugs:
   a csDMARD;
   a bDMARD;
   a tsDMARD;
   one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 nM to Fc [symbol] RI;
(ii) about 30 nM to about 10 μM to Fc [symbol] RIIA_131H;
(iii) about 5 nM to about 5 μM to Fc [symbol] RIIA_131R;
(iv) about 20 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 7.5 pM to about 100 pM to Fc [symbol] RI;
(ii) about 50 nM to about 1 μM to Fc [symbol] RIIA_131H;
(iii) about 5 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 10 nM to about 1 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA 131R, Fc [symbol] RIIb, Fc [symbol] RIIIA_158F and Fc [symbol] RIIIA_158V with an affinity of:
(i) about 6 pM to about 100 pM to Fc [symbol] RI;
(ii) about 50 nM to about 250 nM to Fc [symbol] RIIA_131H;
(iii) about 50 nM to about 250 nM to Fc [symbol] RIIA_131R;
(iv) about 20 nM to about 250 nM to Fc [symbol] RIIb;
(v) about 10 nM to about 250 nM to Fc [symbol] RIIIA_158V; and
(vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 50 pM to about 100 pM to Fc [symbol] RI;
(ii) about 250 nM to about 1 μM to Fc [symbol] RIIA_131H;
(iii) about 50 nM to about 1 μM to Fc [symbol] RIIA_131R;
(iv) about 20 nM to about 1 μM to Fc [symbol] RIIb;
(v) about 10 nM to about 1 μM to Fc [symbol] RIIIA_158V; and
(vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., the antibody binds at least one, at least two, at least three, or at least four of Fc [symbol] RI, Fc [symbol] RIIA_131H, Fc [symbol] RIIA 131R, Fc [symbol] RIIb, Fc [symbol] RIIIA_158F and Fc [symbol] RIIIA_158V with an affinity of:
(i) about 100 pM to Fc [symbol] RI;
(ii) about 900 pM to Fc [symbol] RIIA_131H;
(iii) about 1.5 μM to Fc [symbol] RIIA_131R;
(iv) about 5.2 μM to Fc [symbol] RIIb;
(v) about 1.5 μM to Fc [symbol] RIIIA_158V; and
(vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the antibody does not bind C1q as determined by SPR at 25° C. In some embodiments, the antibody comprises an immunoglobulin constant region. In some embodiments, the immunoglobulin constant region is IgG1. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments the antibody is not peresolimab. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of (i) about 1 pM to about 1 μM to Fc [symbol] RI;
   (ii) about 10 nM to about 10 μM to Fc [symbol] RIIA_131H;
   (iii) about 1 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
a bDMARD;
a tsDMARD;
one or more TNF inhibitors; or
one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 6.0 pM to about 1 μM to Fc [symbol] RI;
   (ii) about 20 nM to about 10 μM to Fc [symbol] RIIA_131H;
   (iii) about 4 nM to about 10 μM to Fc [symbol] RIIA_131R;
   (iv) about 10 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
   (i) about 7.5 pM to about 100 nM to Fc [symbol] RI;
   (ii) about 30 nM to about 10 μM to Fc [symbol] RIIA_131H;
   (iii) about 5 nM to about 5 μM to Fc [symbol] RIIA_131R;
   (iv) about 20 nM to about 10 μM to Fc [symbol] RIIb;
   (v) about 100 pM to about 10 μM to Fc [symbol] RIIIA_158V; and
   (vi) about 10 nM to about 10 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 7.5 pM to about 100 pM to Fc [symbol] RI;
   (ii) about 50 nM to about 1 μM to Fc [symbol] RIIA_131H;
   (iii) about 5 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 10 nM to about 1 μM to Fc [symbol] RIIIA 158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 6 pM to about 100 pM to Fc [symbol] RI;
   (ii) about 50 nM pM to about 250 nM to Fc [symbol] RIIA_131H;
   (iii) about 50 nM to about 250 nM to Fc [symbol] RIIA_131R;
   (iv) about 20 nM to about 250 nM to Fc [symbol] RIIb;
   (v) about 10 nM to about 250 nM to Fc [symbol] RIIIA_158V; and
   (vi) about 50 nM to about 250 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 50 pM to about 100 pM to Fc [symbol] RI;
   (ii) about 250 nM to about 1 μM to Fc [symbol] RIIA_131H;
   (iii) about 50 nM to about 1 μM to Fc [symbol] RIIA_131R;
   (iv) about 20 nM to about 1 μM to Fc [symbol] RIIb;
   (v) about 10 nM to about 1 μM to Fc [symbol] RIIIA_158V; and
   (vi) about 50 nM to about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 100 pM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 1.5 μM to Fc [symbol] RIIIA_158V; and
   (vi) about 500 nM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In another aspect, the present disclosure provides methods of treating autoinflammatory and/or autoimmune diseases comprising administering to a patient in need thereof an effective amount of an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
   (i) about 55 PM to Fc [symbol] RI;
   (ii) about 900 pM to Fc [symbol] RIIA_131H;
   (iii) about 1.5 μM to Fc [symbol] RIIA_131R;
   (iv) about 5.2 μM to Fc [symbol] RIIb;
   (v) about 340 nM to Fc [symbol] RIIIA_158V; and
   (vi) about 1.7 μM to Fc [symbol] RIIIA_158F, respectively, as determined by SPR at 25° C., wherein the binding of the antibody to the ECD does not block binding of human PD-L1 to the ECD and wherein the antibody is a human PD-1 agonist. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In some embodiments, the antibody comprises an immunoglobulin constant region. In some embodiments, the immunoglobulin constant region is IgG1. In some embodiments, the antibody comprises a heavy chain comprising the amino acid sequence of SEQ ID NO: 1 and a light chain comprising the amino acid sequence of SEQ ID NO: 2. In some embodiments, the antibody comprises two heavy chains and two light chains, wherein each heavy chain comprises the amino acid sequence of SEQ ID NO: 1, and each light chain comprises the amino acid sequence of SEQ ID NO: 2. In some embodiments the antibody is not peresolimab. In some embodiments, the autoinflammatory and/or autoimmune disease is RA, SLE, LN, CLE, GCA, vasculitis, UC, CD, SjS, or T1DM and the antibody is administered subcutaneously at about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg Q10W to the patient. In some embodiments, the patient is one who has had an inadequate response to, who has had failure to, or who has been intolerant to one or more of the following drugs:
   a csDMARD;
   a bDMARD;
   a tsDMARD;
   one or more TNF inhibitors; or
   one or more non-TNF inhibitors.

In some embodiments, the present disclosure provides an antibody that binds human PD-1 ECD with an affinity of between about 1 pM and about 100 nM as determined by SPR at 25° C., and binds at least one, at least two, at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of
(i) about 1 pM to about 1 μM to Fc [symbol] RI;
(ii) about 10 nM to about 10 μM to Fc [symbol] RIIA_131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., wherein the binding of the antibody to the ECD does not block binding of human PD-L1 to the ECD, and wherein the antibody further comprises a human IgG1 Fc region variant selected from the group consisting of:
   a) a human IgG1 Fc region variant comprising P247I and A339Q; and
   b) a human IgG1 Fc region variant comprising S298A, E333A, and K334A;
   c) a human IgG1 Fc region variant comprising S239D and I332E;
   d) a human IgG1 Fc region variant comprising S239D, I332E, and A330L;
   e) a human IgG1 Fc region variant comprising G236A, S239D, and I332E; and
   f) a human IgG1 Fc region variant comprising G236A, S239D, I332E, and A330L; and wherein the antibody is a human PD-1 agonist. In some embodiments, binding of the antibody to the ECD does not block binding of human PD-L1 to the ECD. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In other embodiments, the antibody comprises a light chain comprising the amino acid sequence of SEQ ID NO: 2. In other embodiments, the antibody comprises a human IgG1 Fc region variant comprising an amino acid sequence selected from the group consisting of SEQ ID NO: 32, SEQ ID NO: 33, SEQ ID NO: 34, SEQ ID NO: 35, SEQ ID NO: 36, and SEQ ID NO: 37. In some embodiments, the antibody comprises a heavy chain amino acid sequence selected from the group consisting of SEQ ID NO: 38, SEQ ID NO: 39, SEQ ID NO: 40, SEQ ID NO: 41, SEQ ID NO: 42, and SEQ ID NO: 43.

In another aspect, the present disclosure provides an antibody that binds human PD-1 ECD with an affinity of between about 1 pM and about 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and wherein the antibody further comprises a human IgG1 Fc region variant selected from the group consisting of:
   a) a human IgG1 Fc region variant comprising P247I and A339Q; and
   b) a human IgG1 Fc region variant comprising S298A, E333A, and K334A;
   c) a human IgG1 Fc region variant comprising S239D and I332E;
   d) a human IgG1 Fc region variant comprising S239D, I332E, and A330L;
   e) a human IgG1 Fc region variant comprising G236A, S239D, and I332E; and
   f) a human IgG1 Fc region variant comprising G236A, S239D, I332E, and A330L; and wherein the antibody is a human PD-1 agonist. In some embodiments, binding of the antibody to the ECD does not block binding of human PD-L1 to the ECD. In some embodiments, the binding of the antibody to the ECD does not block binding of human PD-L1 or human PD-L2 to the ECD. In some embodiments, the antibody does not bind C1q as determined by SPR at 25° C. In some embodiments, the HCVR of the antibody comprises the amino acid sequence of SEQ ID NO: 3, and the LCVR of the antibody comprises the amino acid sequence of SEQ ID NO: 4. In other embodiments, the antibody comprises a light chain comprising the amino acid sequence of SEQ ID NO: 2. In other embodiments, the antibody comprises a human IgG1 Fc region variant comprising an amino acid sequence selected from the group consisting of SEQ ID NO: 32, SEQ ID NO: 33, SEQ ID NO: 34, SEQ ID NO: 35, SEQ ID NO: 36, and SEQ ID NO: 37. In some embodiments, the antibody comprises a heavy chain amino acid sequence selected from the group consisting of SEQ ID NO: 38, SEQ ID NO: 39, SEQ ID NO: 40, SEQ ID NO: 41, SEQ ID NO: 42, and SEQ ID NO: 43.

### EXAMPLES

The following examples are offered to illustrate, but not to limit, the claimed invention.

### Example 1. PD-1 Agonist Antibodies in Mouse Models of T1DM

Type I diabetes mellitus (T1DM), also called juvenile diabetes or insulin-dependent diabetes, is generally considered a chronic autoimmune disorder in which the body's own immune system attacks the β-cells in the islets of Langerhans of the pancreas, destroying or damaging them sufficiently to reduce and eventually eliminate insulin production. When most of the β-cells have been destroyed, the production of glucagon by the neighboring α-cells is de-repressed. The metabolic consequences of insulin insufficiency and glucagon excess are hyperglycemia and ketoacidosis. T1DM is distinguished from type 2 (non-insulin-dependent) diabetes by the presence of autoantibodies, the genetic link, the insulin dependence and by insulitis, which is characterized by activated T lymphocytes (Van Belle T L, et al., Mouse Models for Type 1 Diabetes. Drug Discov Today Dis Models. 2009 Summer; 6 (2): 41-45)).

### A. RIP-LCMV-GP Mouse Model of TID

Transgenic rat insulin promoter (RIP)-lymphocytic choriomeningitis virus (LCMV)-glycoprotein (GP) mice on the C57B1/6 background have been described previously (see, for example, von Herrath M G, et al., How virus induces a rapid or slow onset insulin-dependent diabetes mellitus in a transgenic model. Immunity, 1994; 1:231-242). LCMV infection of RIP-GP is a well-known widely used virus-induced diabetes model (see, for example, Van Belle, T L, 2009 Summer; 6 (2): 41-45). Using the RIP-LCMV-GP mouse model diabetes is induced by injection of lymphocytic choriomeningitis virus (LCMV).

The in vivo effects of PD-1 agonist antibodies to prevent or reverse diabetes may be tested in the RIP-LCMV-GP mouse model of T1DM. Briefly described, diabetes prevention and reversal may be tested in PD-1 agonist antibody and control treated RIP-LCMV-GP mice. Outcomes of interest include measures of glycemia (i.e., diabetes status), islet histology and quantification of insulitis, quantification of β cell ER stress, apoptosis, and β cell expression of inflammatory markers and selected immune parameters. Insulin and proinsulin content may be measured in islets and serum proinsulin/insulin levels may be measured longitudinally in PD-1 agonist antibody-treated mice as an in vivo and circulating readout of B cell ER stress.

Additionally, or alternatively, blood glucose levels will be analyzed regularly and Kaplan-Meier survival analysis curves generated to determine progression to diabetes in the prevention study and reversal of diabetes.

Additionally, or alternatively, analysis of insulitis and characterization of islet immune infiltrates may be performed using standard immunohistochemistry and multiplex tissue imaging using CODEX technology.

Additionally, or alternatively, morphometric analysis of β and a cell mass and quantification of β cell apoptosis and proliferation. Additionally, or alternatively, this study may include immunofluorescence (IF) for ER stress, inflammatory markers, and HLA H-2Kd (murine Class I) in pancreatic islets, β cell expression of PC1/3, PC2, and CPE by IF, measurement of serum proinsulin/insulin ratios using proinsulin (Alpco) and insulin (Mercodia) ELISAs and/or flow cytometric analysis and/or flow cytometric analysis of immune cell subsets in spleen and pancreatic lymph nodes.

The PD-1 antibodies to be tested in the RIP-LCMV-GP mouse model of T1DM will include surrogates of peresolimab (i.e., Surrogate 1 and Surrogate 2) that bind to mouse PD-1-ECD and have demonstrated the capability to induce PD-1 activity in immunologically relevant contexts (e.g., inhibition of antibody titers in an OVA immunization mouse preclinical model (data not shown)). More specifically, in binding kinetic experiments using a surface plasmon resonance biosensor such as BIAcore™ 2000, BIAcore®3000, BIAcore® 8K, or a BIAcore® T100 (GE Healthcare, Piscataway, NJ), the binding kinetics for peresolimab and antibody Surrogate 1 and antibody Surrogate 2 to human and mouse PD-1-ECD-His, respectively, via SPR at 37° C. (performed essentially as described in WO2019/168745) are as illustrated in Table 1 below.

TABLE 1

| | | | | | KD |
|---|---|---|---|---|---|
| Antibody | Isotype | Analyte | kon (1/Ms) | koff (1/s) | (nM) |
| Surrogate 1 | mIgG2a | mouse PD-1- | 3.06E+5 | 2.37E−4 | 0.77 |
| | | ECD-His | | | |
| Surrogate 2 | mIgG2a | mouse PD-1- | 5.42E+4 | 2.62E−3 | 48.3 |
| | | ECD-His | | | |
| peresolimab | huIgG1 | human PD-1- | 3.07E+5 | 7.01E−4 | 2.29 |
| | | ECD-His* | | | |

*peresolimab does not bind to mouse PD-1 ECD

Mice may be administered isotype control and the test PD-1 antibodies at various doses (e.g., 1-20 mg/kg i.p.). Typically, the drug is administered beginning approximately 1 day prior to LCMV injection and every 4 days through 14 days of follow-up.

Accordingly, anti-mouse PD-1 monoclonal antibody surrogates of peresolimab are expected to show positive effects in vivo on measures of T1DM disease and support the potential of PD-1 agonist antibodies such as peresolimab in preventing or treating T1DM in humans.

### B. Ovalbumin Specific T Cell Transfer Induced Type 1 Diabetes Model

Diabetes may also be induced in mice by injecting both CD4 (OTII) and CD8 (OTI) T cells specific for ovalbumin, isolated from T cell receptor (TCR) OTII and OTI transgenic mice, into a mouse expressing ovalbumin in pancreas (RIP-mOVA mouse). In addition, the mice may be immunized with ovalbumin in complete Freund's adjuvant to induce insulitis more efficiently. The mice may be subcutaneously treated with test agents such as anti-mouse PD-1 monoclonal antibody surrogates of peresolimab, in addition to an isotype control antibody and/or a CD4 depleting antibody as a positive control.

Outcomes of interest include measures of blood glucose (i.e., diabetes status), immune cell phenotyping and quantification in pancreas, islet histology and quantification of insulitis, quantification of β cell ER stress, apoptosis, and β cell expression of inflammatory markers and selected immune parameters. Insulin and proinsulin content may be measured in islets and serum proinsulin/insulin levels may be measured longitudinally in PD-1 agonist antibody-treated mice as an in vivo and circulating readout of β cell ER stress.

Additionally, or alternatively, blood glucose levels may be analyzed regularly and Kaplan-Meier survival analysis curves generated to determine progression to diabetes in the prevention study and reversal of diabetes.

Additionally, or alternatively, analysis of insulitis and characterization of islet immune infiltrates may be performed using standard immunohistochemistry and multiplex tissue imaging using CODEX technology.

### Materials and Methods:

### Mice:

OT-I (strain #003831), OT-II (strain #004194), and RIP-mOVA (strain #005431) mice may be purchased from The Jackson laboratory. Age ranges for mice used are generally 12-16 weeks of age. All animal experiments are performed in compliance with approved institutional guidelines by an animal care and use committee.

### Diabetes Induction:

On Day 0, mice may be weighed and a baseline blood glucose measurement obtained via tail nick using a Contour Next EZ Meter System (Bayer). Mice may be randomly sorted into treatment groups according to baseline glucose numbers and body weight. An emulsion of ovalbumin (Millipore Sigma, A5503) in PBS in a 1:1 ratio with complete Freund's Adjuvant (CFA) may be prepared using an Omni Bead Ruptor Elite homogenizer. Final emulsion may be delivered in 200 μg of ovalbumin in 100 μL total volume. The emulsion may be generally injected subcutaneously at the base of the tail four hours prior to cell injection.

0.2×10⁶ OT-I cells may be combined with 2.0×10⁶ OT-II cells (suspended in PBS) and injected intravenously (200 μL total volume injection). Mice may then be dosed subcutaneously with the appropriate treatment compound (200 μL volume). Compounds may be dosed twice per week.

### Cell Preparation for Diabetes Induction:

For CD4+ cells each GentleMACS C Tube (Miltenyi 130-093-237) may be filled with 5 mL of media (RPMI 1640, 10% FBS (Corning 35-011-CV), sodium pyruvate (Gibco 11360-070), 1 mM 2-mercaptoethanol (Gibco 21985-023), 0.05 mM antibiotic/antimycotic (Gibco 15240-062) and two spleens may be prepared with gentleMACS program “m_spleen_04_01 (rpr: 1260, Duration 59s)”. The cell suspension may be transferred to a 100 micron yellow filter atop of a 50 mL conical tube. The Gentlemacs tubes may be washed with 5 mL of media and rinsed filter. Cell suspensions may be centrifuged at 600×g for 4 minutes at room temperature. The supernatant may be aspirated completely and 1 mL of ACK lysis buffer (Gibco A10492-01) may be used per spleen for red Blood Cell Lysis. After incubating at room temperature for 1 minute, samples may be neutralized with 10 mL of media and filtered through a new 100 μm yellow filter over a 50 ml conical tube. The cells may be centrifuged at 600×g for 4 min at 4° C. and resuspended in 5 mL of media. After combining contents of all tubes the cells may be centrifuged at 500×g for 5 minutes at 4° C. and then resuspended in 45 mL media. The cells may then be stored on ice followed by a cell count and CD4+ T Cell isolation using CD4+ T Cell negative selection Isolation Kit (Miltenyi 130-104-454) protocol.

For CD8a⁺ cells, a 100 micron strainer may be placed atop a 50 ml conical tube and rinsed with 2 mL media (RPMI 1640, 10% FBS (Corning 35-011-CV), Sodium Pyruvate (Gibco 11360-070), 1 mM 2-mercaptoethanol (Gibco 21985-023), 0.05 mM Antibiotic/Antimycotic (Gibco 15240-062). One spleen may be placed into the cell strainer and a plunger from a sterile 3 mL syringe may be used to crush the spleen and push through the strainer. The cell strainer and plunger may be rinsed with 10 mL media repeat with fresh strainer for each spleen for a total of 3 spleens per tube. The total volume may be brought to 45 mL and stored on ice until all spleens are processed. The cell suspension may be centrifuged for 5 min at 230× g. After removing the supernatant, red blood cells may be lysed using 1 mL of ACK lysing buffer (Gibco A10492-01) per each spleen mix and incubating for 1 min at room temperature. Media may be added to bring to 20 mL. After centrifugation at 250×g for 7 minutes, the supernatant may be decanted. All cells may be combined after addition of 5 mL per tube to bring the total volume to 40 mL medium and washed for 5 min at 230× g to eliminate proteases and enzymes that may damage the cells for a total of 3 times. Next, the cells may be filtered through a 70 μm cell strainer (Fisher, 22363548) and stored on ice while conducting cell count and CD8+ T Cell isolation using CD8+ T Cell negative selection Isolation Kit (Miltenyi 130-104-075) protocol.

Isolated cells may be washed twice in PBS without calcium and magnesium (Corning 21-031-CV), counted and calculated the cell number and volume for the cell density of 2×10⁵ cells/mL of CD8+ OTI cells and 2×10⁶ cells/mL of CD4+ OTII cells for injection of 200 μL in vivo.

### Necropsy:

On Day 8, 400 μl of whole blood may be collected in a serum separator tube and centrifuged at 10,000×g for five minutes to obtain serum. Mice are humanely euthanized. Spleen and pancreas may be removed and weighed. Half of the pancreas may be submitted for flow cytometry or FACs analysis.

### Cell Preparation for Flow Cytometry:

Complete RPMI medium (RPMI 1640 (Corning 10-041-CV), 5% FBS (Corning 35-011-CV), 1% Glutamax (Gibco 35050-061), 2 mM EDTA (0.5M stock, Ambion AM92606)) may be used throughout the single cell isolation of pancreatic cells using Slide method. Two mL of media may be added to a tissue culture dish (Corning 430166). A single pancreas may be placed on the frosted side a microslide (Corning 2948) and using another slide the tissue may be dissociated by gentle rubbing of the frosted side of both slides. Slides may be washed into the dish containing the dissociated cells and full volume from the dish was tan through the 50 ml conical tube with the 100 micron cell strainer (Cell Treat, 229486). The back of a syringe may be used to crush the remaining tissue through the cell strainer. The dish may be washed with 5 mL of media and the contents transferred to the same cell strainer to rinse with 5-10 mL media before discarding. Media may be added to tubes as needed to bring the total volume on all conical tubes to 25-30 mL. Cells may be centrifuged at 500×g for 5 minutes at 4° C. and resuspended in 2 mL media and transferred to a deep well 96 well plate. 200 μl of each cell suspension may be transferred to a corresponding 96 well filter plate (PALL 8027) and centrifuged at 500×g for 5 minutes at 4° C. 100 μL of the filtered cells may be used for FACS panel staining. 20 μL of the filtered cells may be used for additional cell counting plate.

### Flow Cytometry Staining:

Cells may be blocked in 100 μL of FACS buffer (BD 554656) containing Human truStain FcX (Biolegend 422302) for 10 minutes, centrifuged at 1200 rpm for 5 minutes. The blocking buffer may be removed and 100 μL of antibody cocktail may be added [CD4-APC (clone RM4-5, BioLegend 100516), CD8-AF700 (clone 53-6.7, BioLegend 100730), Vβ5-FITC (clone MR9-4, BioLegend 139514), CD44-BV421 (clone IM7, BioLegend 103039), CD62L-BV785 (clone MEL-14, BioLegend 104440), CD69-BV605 (clone H1 2F3, BioLegend 104529), PD-1-PE (clone 29F.1A, 1 BioLegend 135205), Viability Zombie Aqua (Biolegend 77143)] to each well and stained for 30 minutes on ice. Cells may be washed and then fixed using Cytofix (BD 554655). CountBright Plus absolute counting Beads (Invitrogen, Cat C36995, Invitrogen) may be added and analyzed on Fortessa X-20 for FACS read out. Data analysis may be done using FlowJo software.

In experiments performed essentially as described above for the Ovalbumin specific T cell transfer induced Type 1 Diabetes Model treatment groups for the assay were set up as the following: (1) mIgG2a isotype control (10 mg/kg), (2) Surrogate 2 PD-1 agonist antibody described above in Table 1 (10 mg/kg), (3) anti-CD4 (10 mg/kg), and (4) non-carrier negative control RIP-mOVA mice (10 mg/kg mIgG2a isotype control). Glucose measurements were obtained on Day 0, 3, and Days 5-8. The mice were sacrificed on day 8 and the pancreas was processed for single cell analysis to quantitate CD8 TCR Vβ5 chain (OTI TCR) T effector cell and T regulatory cell infiltration into pancreas.

Results show a statistically significant reduction of blood glucose area under curve (AUC) over days 6-8 by Surrogate 2 PD-1 agonist antibody (60% reduction) compared to isotype control antibody (see, Table 2). Group differences were assessed using one-way ANOVA followed by Dunnett's test for multiple comparisons using log transformed data (p <0.05). In addition, the number of CD8 TCR Vβ chain (OTI TCR) effector memory cells was significantly reduced in pancreas on day 8 by Surrogate 2 antibody as compared to the isotype control antibody (see Table 3).

TABLE 2
% Inhibition Glucose (AUC Days 6-8)

| Treatment | Mean % | SEM |
|---|---|---|
| Isotype control | 0 | 6 |
| Surrogate 2 antibody | 60 | 10 |
| CD4 Depleting antibody | 56 | 16 |

TABLE 3
Pancreas % CD8 Vβ5+ Effector Memory Cell Effector

| Treatment | Mean % | SEM |
|---|---|---|
| Isotype control | 100 | 3.6 |
| Surrogate 2 antibody | 15.6 | 0.7 |
| CD4 Depleting antibody | 44.7 | 8.2 |

TABLE 4
Pancreas % CD44+ T regulatory cells from CD44+ CD4+ T cells

| Treatment | Mean % | SEM |
|---|---|---|
| Isotype control | 6.6 | 0.3 |
| Surrogate 2 antibody | 11.2 | 0.4 |
| CD4 Depleting antibody | 0 | 0 |

### C. T Cell Transfer Induced Inflammatory Bowel Disease Model

Colitis may be induced in mice by injecting CD4 T cells from C57BL/6 mice to a syngeneic lymphocyte deficient Rag2 knock out mice. The mice may be subcutaneously treated with test agents such as anti-PD-1 monoclonal antibody surrogates of peresolimab, in addition to an isotype control antibody and/or an IL-23 neutralizing antibody. Outcomes of interest include immune cell infiltration in the intestine.

### Materials and Methods:

### Mice:

Donor mice-female C57BL/6 mice (strain #000664) may be purchased from The Jackson Laboratory. Age ranges for mice used are generally 10 weeks. Recipient mice-female Rag2 KO mice (strain #RAGN12) may be purchased from Taconic Biosciences. Age ranges for mice used are generally 10 weeks. All animal experiment should be performed in compliance with approved institutional guidelines by an animal care and use committee.

### Colitis Induction:

On Day 0, CD4+ T cells may be isolated from donor mice spleens, stained, and sorted for CD45RBhigh/CD44+ (naïve T) cells and CD45RBlow/CD44+ (Treg) cells (CD45RBhigh and CD45RBlow fractions may be operationally defined as the upper 40% and lower 10% to 20%, respectively, of CD45RB-staining CD4+ cells) using cell sorters. 4×10⁵ CD45RBhigh/CD44+ cells or 4×10⁵ CD45RBlow/CD44+ cells in 200 μL injection volume may be injected intraperitoneally into respective recipient mice. Mice may then be dosed subcutaneously with the appropriate treatment compound (10 mg/kg with 200 UL injection volume or 0.3 mg/kg IL-23 antibody only). Compounds may be dosed once a week or twice per week (IL-23 Antibody only).

### Cell Preparation for Colitis Induction:

For CD45RBlow/CD44″ cell isolation each GentleMACS C Tube (Milteney 130-093-237) may be filled with 6 mL media (1×DPBS containing 2% FBS and 1 mM EDTA (10 ml FBS+1 ml 0.5 M EDTA per 500 ml DPBS without calcium and magnesium) and 3 spleens. Processing may be done with gentalMACs program m_spleen_04. The sample may be centrifuged for 30 seconds and the sample material collected at the bottom of the tube. The sample may be resuspend in 6 mL media. The cell suspension may be applied to a 100 μm filter placed on a 50 mL tube. The filter may be washed with 5 mL of medium. The cell suspension may be centrifuged at 300×g for 10 minutes at room temperature. The supernatant may be aspirated and the cells may be resuspended with medium at 1×10⁸ cells/ml, and the cells may be transferred in a new tube. The T cells may be isolated using an EasySep Mouse CD4+ T Cell Isolation Kit (STEMCELL Technologies, #19852) based on manufacturer's protocol. Isolated cells may be counted using the Cellometer Auto 2000 and prepared for FACS staining. The cells may be centrifuged at 400×g for 8 minutes at 4° C. The supernatant may be aspirated. 1 mL of FACS buffer (1×DPBS containing 2% FBS, 25 mM HEPES buffer, and 1 mM EDTA (10 mL FBS+12.5 mL 1M HEPES buffer+1 mL 0.5 M EDTA per 500 ml DPBS without calcium and magnesium) may be added to the tube. Staining antibodies may be added, with 0.6 μL of CD4-PE PE (Thermo Fisher Scientific, #12-0042), 0.4 μL of CD44-APC PE (Thermo Fisher Scientific, #12-0042), and 1 μL of CD45RB-FITC (Thermo Fisher Scientific, #11-0455). The tubes may be resuspended and incubated for 15 minutes at 4° C. in the dark. FACS buffer may be added to top up to 20 mL and then the tubes may be centrifuged at 400×g for 8 minutes at 4° C. The supernatant may be aspirated and cells may be resuspended with FACS buffer at 50×10⁶ cells/ml. 50 μl of diluted viability stain SYTOX blue (10 μl SYTOX blue+490 μl DPBS) may be added per 1 ml cell suspension. Cells may be distributed 200×10⁶ cells into a 40 μm filter capped FACS tube. 15 mL collection tubes may be prepared with 0.5 mL of 100% FBS on ice. The cells may be sorted to CD45RBlow/CD44+ and CD45RBhigh/CD44+/−utilizing a high-speed cell sorting cytometer (Becton Dickinson FACSAria Fusion) equipped with 5 lasers (355 nm, 405 nm, 488 nm, 561 nm, 633 nm) and acquisition and analysis software (BD FACSDiva, v9.0.1). Flow cytometry analysis of cell suspensions may be analyzed at a drop drive frequency of 87 kHz and flow rates up to 20 k/sec. Unwanted fluorescence from a primary channel that is detected in a secondary channel may be compensated for via mathematical subtraction. Final gating criteria for sorted populations may be based on initial gates to exclude dead cells and eliminate doublets determined by scatter geometry. Sort precision may be set to achieve the highest purity for target populations. Isolated cells may be collected into 15 mL conical tubes containing 0.5 ml of 100% FBS, which may be kept on ice, before centrifugation.

The FACS sorted CD4 T cells may be centrifuged at 400×g for 8 minutes at 4° C. The supernatant may be aspirated and the cells may be resuspend with DPBS. The cell suspension may be transferred to a clean tube to combine everything. The collection tubes may be washed with DPBS to collect all cells and minimize cell loss. The cells may be diluted 1:10 in DPBS, count using the Cellometer Auto 2000, and the required DPBS volume may be added to achieve 400,000 CD45RBhigh/CD44+ cells or CD45RBlow/CD44+ cells per 200 μL injection.

### Necropsy:

On Day 52, 400 mL of whole blood may be collected and placed in a serum separator tube and centrifuged at 10,000×g for five minutes to obtain serum. Mice are humanely euthanized. Colon may be removed from the body and stools may be gently removed, using a cotton tipped applicator. Intraepithelial lymphocytes (IEL) may be collected by cutting the intestine first longitudinally, followed by a wash in ice-cold DPBS, and cutting laterally into pieces of 0.5-1 cm length. The tissue pieces may be transferred into a 50 mL tube containing 10 mL of Buffer (500 ml HBSS/5% FBS/2 mM EDTA/0.15 mg/ml (1 mM) DTT/10 mM HEPES). The samples may be incubated for 15 minutes at 37° C. shaking at 250 rpm. The samples may be vortexed for 30 seconds. The debris may be allowed to fall to the bottom of the tube. The cells may be passed through a 100 μm yellow cell strainer. The flow-through cells (intraepithelial lymphocytes, IELs) may be transferred into a 15 mL tube and stored on ice. The cells may be submitted for flow cytometry or FACS analysis.

### Flow Cytometry:

Cells may be blocked in 50 μL of FACS buffer (BD 554656) containing Fc Block (Clone 2.4G2, BD, Cat 553142), for 15 minutes at 4° C. Then 50 μL of antibody cocktail may be added (CD45 BV605 (Clone 30-F11, BD, Cat 563053), Ter119 APC/Cy7 (Clone TER-119, BioLegend, Cat 116222), Fixable Live/Dead NIR (ThermoFisher, Cat L10119), F4/80 BV421 (Clone BM8, BioLegend, Cat 123137), CD11b BV785 (Clone M1/70, BioLegend, Cat 101243), Ly6g PerCP/Cy5.5 (Clone 1A8, BioLegend, Cat 127615), B220 PE/Cy5 (Clone RA3-6B2, BioLegend, Cat 103210), CD3 FITC (clone 145-2C11, BioLegend, Cat 100306), PDL-2 PE (Clone Ty25, BioLegend, Cat 107205), CD38 PE/Cy7 (Clone 90, BioLegend, Cat 102718), CD86 AF647 (Clone GL-1, BioLegend, Cat 105020) to each well and stained for 30 minutes on ice. Cells may be washed and then fixed using Cytofix (BD 554655). CountBright Plus absolute counting Beads (Invitrogen, Cat C36995, Invitrogen) may be added and analyzed on Fortessa X-20 for FACS read out. Data analysis may be done using FlowJo software. Percent control may be calculated using the formula

$$[ ( Treatment - 10 mg / kg mIgG 2 a s . c . BIW ; Tregs ) \text{} / ( 10 mg / kg mIgG 2 a s . c . BIW ; naïve T cells - 10 mg / kg mIgG 2 a s . c . BIW ; Tregs ) ] \times 100.$$

### Results:

In experiments performed essentially as described above for the T cell transfer induced Inflammatory Bowel Disease Model assay, results showed a statistically significant reduction of CD45+ hematopoietic cells, neutrophils and CD3+ T cells in intestine by Surrogate 2 PD-1 agonist antibody (58.1%, 63.9% and 67%, respectively) and anti-IL23 antibody (78.6%, 79.8% and 71.4%, respectively) compared to isotype control antibody (data not shown). Group differences were assessed using one-way ANOVA using Dunnett's multiple comparison test.

### Example 2. A Phase 2(a) Study of Peresolimab in Participants with Moderately-to-Severely Active RA

### Clinical Trial Design:

A Phase 2, placebo-controlled, double-blind, randomized study in adult participants with moderately-to-severely active RA who have had an inadequate response to csDMARDs (i.e., csDMARD-IR), or to bDMARDs/tsDMARDs (i.e., bDMARD/tsDMARD-IR) may be conducted to evaluate the safety and efficacy of peresolimab (as compared to placebo) when administered intravenously (either 300 mg or 700 mg) Q4W.

Participants who fulfilled the eligibility criteria described below entered a double-blind treatment period (Period 1) and were randomized to receive peresolimab 700 mg, peresolimab 300 mg, or placebo in an allocation ratio of 2:1:1. The double-blind treatment period established the clinical efficacy and safety of peresolimab up to 12 weeks. The study was evaluated for the primary objective (i.e., a significantly greater change from baseline at Week 12 in DAS28-CRP score in participants treated with peresolimab as compared to participants treated with placebo at both tested doses (300 mg and 700 mg)) at the end of the double-blind treatment period.

Following the double-blind treatment period, participants were able to continue into Period 2 of the trial. In Period 2, participants assigned to peresolimab at baseline, and achieving low disease activity (LDA) (i.e., CDAI≤10) at Week 14, could continue to receive peresolimab to assess safety and tolerability data and evaluate clinical activity with additional dosing up to 24 weeks, for example. All other participants at Week 14, regardless of baseline treatment assignment, received standard of care treatment at the investigator's discretion. After Period 2, participants were followed post-treatment for 12 weeks to assess safety, study drug exposure, and clinical disease activity measures.

Secondary objectives of the study included evaluation of the effect of peresolimab compared to placebo for measures of disease activity such as:
Proportion of participants achieving ACR50 or ACR70 at Week 12
Proportion of participants achieving LDA or remission at Week 12 for
   DAS28-CRP or DAS28-hsCRP,
   DAS28-ESR,
   SDAI, and
   CDAI.
Change from baseline at Week 12 for mean
   DAS28-CRP or DAS28-hsCRP,
   SDAI, and
   CDAI.
Change from baseline at Week 12 for ACR core set values
   68 tender joint count
   66 swollen joint count, and
   Physician's Global Assessment of Disease Activity (VAS).

Secondary objectives of the study also included evaluation of the effect of peresolimab compared to placebo for patient reported outcome measure such as:
Change from baseline at Week 12 for patient-reported ACR core set values
   Patient's Global Assessment of Disease Activity (VAS)
   Patient's Assessment of Arthritis Pain (VAS), and
   patient's assessment of physical function using HAQ-DI.
Change from baseline at Week 12 for
   the duration and severity of morning joint stiffness
   Functional Assessment of Chronic Illness Therapy-Fatigue (FACIT F) scores
Change from baseline at Week 12 for
   SF-36 domains
   SF-36 Physical Component Summary, and
   SF-36 Mental Component Summary.

Secondary objectives of the study also included evaluation of the effect of peresolimab for measures of disease activity such as:
Proportion of participants achieving LDA or remission at Weeks 24 and 60 for
   DAS28-CRP or DAS28-hsCRP,
   DAS28-ESR,
   SDAI, and
   CDAI.
Proportion of participants achieving ACR20, ACR50, or ACR70 at Weeks 24 and 60.
Change from baseline at Weeks 24 and 60 for ACR core set values
   68 tender joint count
   66 swollen joint count, and
   Physician's Global Assessment of Disease Activity (VAS).
Change from baseline at Weeks 24 and 60 for mean
   DAS28-CRP or DAS28-hsCRP,
   SDAI, and
   CDAI.

Secondary objectives of the study also included characterization of the pharmacokinetics of peresolimab such as the observed trough drug concentration at Week 12.

### Participant Eligibility:

Participants were considered eligible for inclusion in the study described above in this Example 2 if they were ≥18 years of age, had a diagnosis of adult onset RA as defined by the 2010 American College of Rheumatology (ACR)/European League Against Rheumatism (EULAR) classification criteria (see, for example, Aletaha, D., et al., 2010 rheumatoid arthritis classification criteria: an American College of Rheumatology European League Against Rheumatism collaborative initiative. Ann Rheum Dis, 2010. 69 (9): p. 1580-8) for at least three months prior to screening, had moderately-to-severely active RA defined by the presence of ≥6 swollen joints (based on 66 joint count) and ≥6 tender joints (based on 68 joint count) at screening and baseline, and had demonstrated an inadequate response to, loss of response, or intolerance to at least one csDMARD treatment or at least one bDMARD/tsDMARD treatment.

### Drug Product and Formulation:

The drug product is composed of peresolimab supplied as a sterile solution formulation in glass vials at 50-100 mg/mL and the excipients histidine (5 mM), sucrose (8%), and polysorbate 80 (0.02% PS80), pH 6.0. For IV infusion, the drug product solution is further diluted with sodium chloride for injection, US Pharmacopeia or sterile diluent (containing the inactive ingredients sodium chloride, polysorbate 80, and citrate).

### Concomitant Therapies:

Additional drugs should be avoided during the study unless required to treat an adverse event or for the treatment of an ongoing medical condition. Investigators may be instructed to follow local guidelines for the management of lipid disorders. If the need for other concomitant medications arises, discontinuation of the participant from study intervention or the study was at the discretion of the investigator in consultation with sponsor (or designee).

### Results:

In the Phase 2, placebo-controlled, double-blind, randomized study in adult participants with moderately-to-severely active RA who have had an inadequate response to csDMARDs, or to bDMARDs/tsDMARDs conducted essentially as described above in this Example 2.

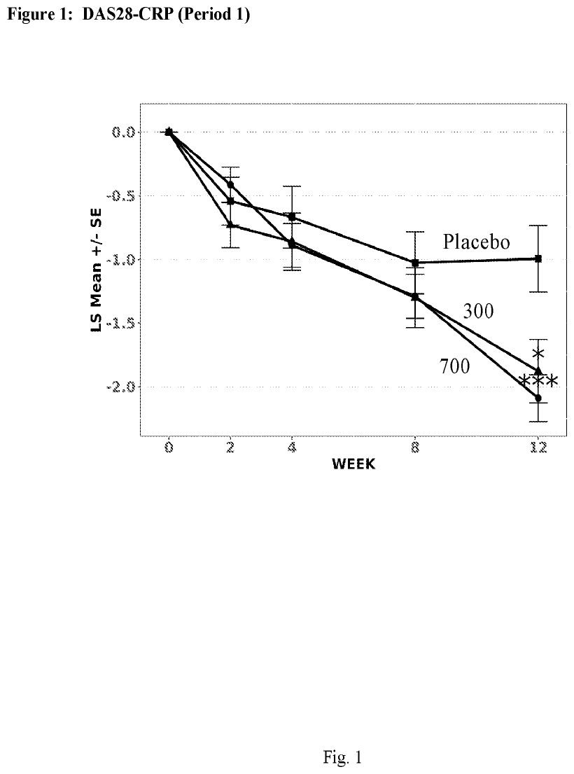

Treatment comparison vs. placebo for the primary endpoint was made using mixed-effects model of repeated measures and logistic regression model for binary endpoints. No adjustment for multiplicity was made; for assessments beyond the primary endpoint, nominal p-values are reported. The safety and efficacy of peresolimab (as compared to placebo) when administered intravenously (IV) (either 300 mg or 700 mg) Q4W was demonstrated. More specifically, there were significantly greater improvement in participants treated with peresolimab 300 mg (Least Square Mean [LSM]=−1.88; CI=−2.37, −1.738; p=0.017) and peresolimab 700 mg (LSM=−2.4409; CI=−2.46, −1.72; p<0.001), versus placebo (LSM=−0.99; CI=−1.51, −0.47) in the primary efficacy outcome of DAS28-CRP CFB at Week 12. Therefore, this clinical trial met its primary endpoint of a significantly greater change from baseline at Week 12 in DAS28-CRP score in participants treated with peresolimab vs participants treated with placebo at both tested doses (700 mg [p value<0.001] and 300 mg [p value<0.05]) (FIG. 1).

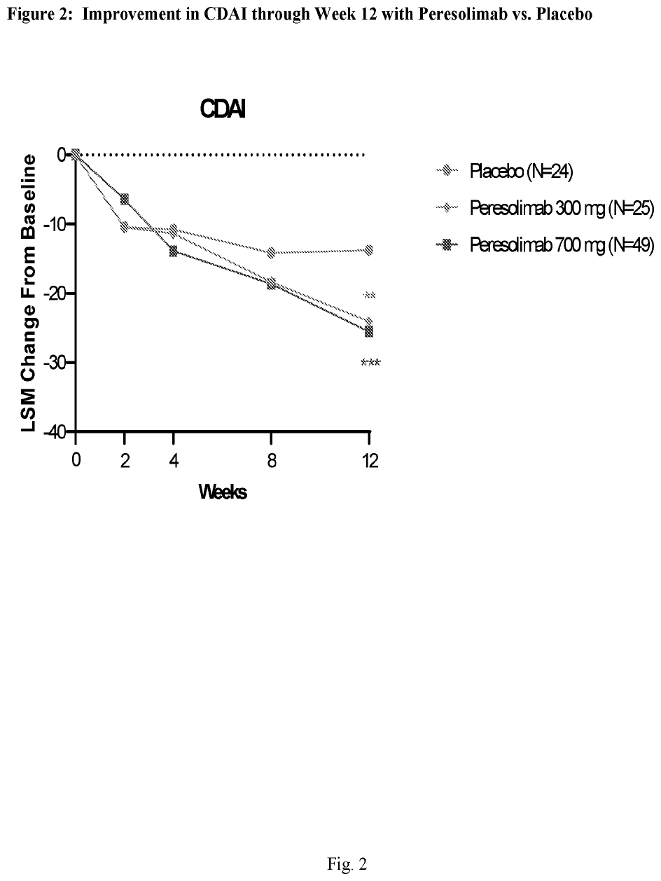

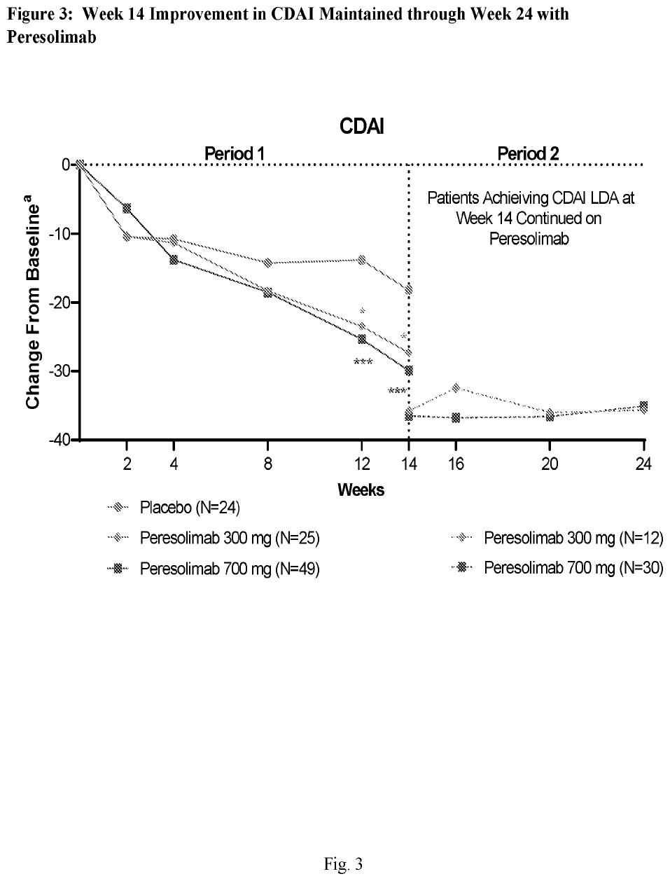

Significant improvements were also seen in CDAI change from baseline between participants treated with either peresolimab dose (p value <0.01 for both doses) and placebo (FIG. 2), and efficacy was maintained through week 24 in most patients achieving CDAI low disease activity by Week 14 (FIG. 3). Furthermore, a significantly greater percentage of participants treated with peresolimab 700 mg achieved ACR20 (p value <0.05) compared to participants treated with placebo by Week 12. More specifically, at Week 12, 71.4% of participants treated with 700 mg peresolimab achieved ACR20 compared to 41.7% of participants treated with placebo). Additionally, peresolimab surprisingly showed at least comparable efficacy in bDMARD/tsDMARD-IR patients as compared to csDMARD-IR patients.

Peresolimab appeared to favor placebo for several secondary endpoints at Week 12 as well. More specifically, there were significant increases between participants treated with peresolimab 700 mg versus placebo in CDAI CFB (p<0.001), SDAI CFB (p<0.001), proportion of participants achieving ACR20 (p=0.025), TJC CFB (p=0.017), SJC CFB (p=0.002), PGA CFB (p=0.038) and Pain CFB (p=0.031) at Week 12 (data not shown). Significant increases were also found with peresolimab 300 mg versus placebo in CDAI CFB (p<=0.008), SDAI CFB (p=0.003), TJC CFP (p=0.09), and SJC CFB (p=0.011) at Week 12 (data not shown).

No obvious trends in any type of AEs, no dose-dependence of AEs, no peresolimab related SAE's and no infusion reactions across dose cohorts through Week 24 were observed. Therefore, peresolimab also showed a favorable safety profile.

Briefly stated, the results of the study described in this Example 2 are the first meaningful evidence of clinical safety and efficacy for a PD-1 agonist in autoimmune disease and the first such result for a checkpoint agonist in rheumatology. Additionally, the study described in this Example 2 is the first clinical trial to demonstrate efficacy of a PD-1 agonist antibody in patients with RA who have failed prior csDMARD therapy alone, or failed b/tsDMARD therapy. Therefore, peresolimab may address unmet needs for patients with RA who were refractory to or intolerant of DMARDs, which are typically used as first line treatments in patients with RA. Also, contrary to what has been typically observed in patients with RA, the more refractory population surprisingly seems to have greater numerical improvement in disease activity following peresolimab treatment relative to the b/tsDMARD naive group. This is likely due to the novel mechanism of action employed by peresolimab, which may allow the treatment methods disclosed herein to address unmet clinical needs for patients with RA, including maintaining efficacy in subgroups of patients who have previously failed first line treatments.

### Example 3. A Phase 2 (b) Study of Peresolimab in Participants with Moderately-to-Severely Active RA

### Rationale and Objectives

The purpose of this clinical study is to evaluate the safety and efficacy of peresolimab in patients with moderately-to-severely active RA (see, Trial Registration NCT05516758).

### Primary Outcome Measure:

Percentage of participants achieving American College of Rheumatology (ACR) 20-20% improvement in the ACR core set values at Week 12.

### Secondary Outcome Measures:

Secondary objectives of the study include evaluation of the effect of peresolimab compared to placebo for measures of disease activity such as:
Proportion of participants achieving ACR50 or ACR70 at Week 12
Proportion of participants achieving LDA or remission at Week 12 for
   DAS28-CRP or DAS28-hsCRP,
   DAS28-ESR,
   SDAI, and
   CDAI.
Change from baseline at Week 12 for mean
   DAS28-CRP or DAS28-hsCRP,
   SDAI, and
   CDAI.
Change from baseline at Week 12 for ACR core set values
   68 tender joint count,
   66 swollen joint count, and
   Physician's Global Assessment of Disease Activity (VAS).

Secondary objectives of the study also include evaluation of the effect of peresolimab compared to placebo for patient reported outcome measure such as:
Change from baseline at Week 12 for patient-reported ACR core set values
   Patient's Global Assessment of Disease Activity (VAS)
   Patient's Assessment of Arthritis Pain (VAS), and
   patient's assessment of physical function using HAQ-DI.
Change from baseline at Week 12 for
   the duration and severity of morning joint stiffness
   Functional Assessment of Chronic Illness Therapy-Fatigue (FACIT F) scores
Change from baseline at Week 12 for
   SF-36 domains
   SF-36 Physical Component Summary, and
   SF-36 Mental Component Summary.

Secondary objectives of the study also include evaluation of the effect of peresolimab for measures of disease activity such as:
Proportion of participants achieving LDA or remission at Weeks 24 and 60 for
   DAS28-CRP or DAS28-hsCRP,
   DAS28-ESR,
   SDAI, and
   CDAI.
Proportion of participants achieving ACR20, ACR50, or ACR70 at Weeks 24 and 60.
Change from baseline at Weeks 24 and 60 for ACR core set values
   68 tender joint count
   66 swollen joint count, and
   Physician's Global Assessment of Disease Activity (VAS).
Change from baseline at Weeks 24 and 60 for mean
   DAS28-CRP or DAS28-hsCRP,
   SDAI, and
   CDAI.

Secondary objectives of the study also include characterization of the pharmacokinetics of peresolimab such as the observed trough drug concentration at Week 12.

### Clinical Trial Design:

In general, an individual may take part in this study if they
Are 18 years of age or older at the time of signing the informed consent.
Were diagnosed with RA as an adult, at least 3 months prior to screening, as defined by the 2010 American College of Rheumatology (ACR)/European League Against Rheumatism (EULAR) classification criteria.
Currently have moderately-to-severely active RA.
Have positive test results for rheumatoid factor or anti-citrullinated peptide antibodies at screening OR previous radiographs that show images of the hands or feet consistent with RA.
Have C-reactive protein (CRP)>1.2 times upper limit of normal (ULN).
Have active synovitis in ≥1 joint in hands or wrists at screening determined by MRI.
Have up to date vaccinations per regional and national guidelines, specifically influenza, pneumonia, and zoster.
Have tried at least 1 of these treatments for their RA before, but continued to have signs and symptoms of active RA:
   azathioprine
   methotrexate
   hydroxychloroquine
   leflunomide
   sulfasalazine
   bDMARDs, or
   tsDMARDs.

Adult RA patients will be randomized to receive 100 mg (60 patients), 400 mg (120 patients), 1000 mg (120 patients) of peresolimab subcutaneously once every 4 weeks (SC Q4W), or placebo Q4W (120 patients). Patients who are randomized to placebo at Week 0 will be randomized to receive 1000 mg, or 400 mg of peresolimab SC Q4W at Week 12.

Participants receiving 400 mg or 1000 mg of peresolimab will continue their assigned dose and dosing frequency until Week 24. At Week 24, participants may be switched to Q12W dosing following assessment of CDAI scores.

| If, at Week 24, a | |
|---|---|
| participant has a | |
| CDAI score of . . . | Then . . . |
| >10 | they will continue the same assigned dose |
| | and dosing frequency for the remaining |
| | duration of the treatment period. |
| ≤10 | they will continue the same assigned dose |
| | for the duration of the treatment period, |
| | and will be randomly assigned to either |
| | continue the Q4W dosing frequency, or |
| | switch to the Q12W dosing frequency. |

The efficacy of the RA treatment (i.e. therapeutic effect) can be determined by various parameters, including, but not limiting to, ACR 20, 50 and 70 response over time, ACR-N index of improvement over time, DAS28 (using CRP and ESR) score over time, proportion of subjects with EULAR response over time, proportion of subjects in remission over time (making use of following definitions: DAS28, CDAI, SDAI, Boolean remission), including inhibition of structural damages, change from baseline in disease activity over time (making use of following disease activity scores (DAS28, SDAI, CDAI), change from baseline in HAQ-DI over time, the proportion of HAQ-DI responders over time, the changes from baseline in the physical and mental component scores of the SF-36, the change from baseline in FACIT-F, duration of morning stiffness. The ACR 20, 50 and 70 response criteria are an endpoint that indicates the proportion of patients with at least a 20 percent (50 percent or 70 percent) improvement in the number of swollen and tender joints. A patient will be considered a responder according to ACR20 criteria if he/she has:
at least 20 percent improvement in the two following measures:
   tender 28-joint count
   swollen 28-joint count; and
at least 20% improvement in at least 3 of the following 5 measures:
   Patient's assessment of RA pain (VAS 100 mm)
   Patient's global assessment of disease activity (VAS 100 mm)
   Physician's global assessment of disease activity VAS 100 mm)
   Patient self-assessed disability (Health Assessment Questionnaire score)
   Acute phase reactant (hs-CRP or erythrocyte sedimentation rate (ESR)).

The dose levels and regimens are based on safety, PK, target engagement, and efficacy data from Studies JIA-MC KDAB (i.e., NCT03715192), J1A-MC-KDAC (i.e., NCT04152382), and J1A-MC-KDAD (i.e., NCT04634253), nonclinical toxicology data, and PK/PD modeling and simulations.

The dose levels and regimens described herein are anticipated to provide an exposure range that is pharmacologically active and cover a range in clinical response.

The dosing regimens for Weeks 0 to 24 are 100, 400, and 1000 mg SC Q4W. The doses of 400 and 1000 mg SC Q4W are projected to provide similar to slightly lower projected exposure range as the efficacious regimens of 300 and 700 mg IV Q4W evaluated in Study KDAD, based on SC bioavailability of approximately 60% from Study KDAB.

The dose of 100 mg SC Q4W was selected to provide data for a lower exposure range to explore the relationship of peresolimab exposure to clinical response. Receptor occupancy data were used as a surrogate for pharmacological activity to help assess dose levels and dosing frequencies. Data from Study KDAB and modeling suggests that 100 mg SC Q4W would provide high target engagement in the peripheral blood over the 4-week dosing interval.

The projected exposure range for the selected dosing regimens have an acceptable safety profile in healthy participants in the single-dose Study KDAB, participants with psoriasis in the multiple-dose Study KDAC, and participants with RA in the multiple-dose Study KDAD.

From available human PK and preclinical monkey toxicology data, the estimated margins of safety for the highest dose of 1000 mg SC Q4W are 7.2 based on dose, and 64.2 based on exposure.

At Week 24, participants who were receiving 400 mg or 1000 mg Q4W peresolimab and have a CDAI score of ≤10 may be randomly assigned to a Q12W dosing frequency. Receptor occupancy data from Study KDAB show that a single dose of 350 mg or 700 mg IV produced high target engagement in the peripheral blood over approximately 12 weeks, whereas lower dose levels provided coverage for shorter durations.

### Drug Product and Formulation:

The drug product is composed of peresolimab supplied as a sterile solution formulation in glass vials at 50-100 mg/mL and the excipients histidine (5 mM), sucrose (8%), and polysorbate 80 (0.02%), pH 6.0. For SC injection, the 50 mg/mL drug product solution is administered as is. For IV infusion, the drug product solution is further diluted with sodium chloride for injection, US Pharmacopeia or sterile diluent (containing the inactive ingredients sodium chloride, polysorbate 80, and citrate).

### Example 4: PD-1 Antibody Agonists Inhibition of T Cell Receptor Activation Induced NFAT Signaling is Dependent Upon FcγR Engagement

NFAT (Nuclear factor of activated T cells) is a transcription factor that plays an important role in mediating T cell receptor activation to immune response by modulating expression of genes encoding inflammatory cytokines, e.g., TNF, IL-2 as well as several cell surface receptors. The ability of PD-1 agonistic antibodies such as, but not limited to, peresolimab and variants thereof, to inhibit T cell receptor activation induced NFAT signaling or otherwise can be measured as follows:

Jurkat T cells transfected with both NFAT luciferase reporter and human PD-1 (Promega) are activated with human CD3 antibody and peripheral blood mononuclear cells (PBMCs) or THP-1 cells (ATCCR) or CHO cells transfected with FcγRIIB. Briefly described, human peripheral blood mononuclear cells (PBMCs) are isolated from healthy human Trima LRS (San Diego Blood Bank; San Diego, Calif.) using Ficoll density gradient centrifugation method by overlaying 35 mL diluted blood (tenfold dilution in PBS without calcium or magnesium onto 15 mL 37° C. Ficoll (Ficoll-Paque Plus, GE Healthcare; Ser. No. 17/114,4002) in 50 mL conical tubes. Tubes are centrifuged at room temperature for 30 minutes at 900×G. The cell interface is transferred to a new 50 ml conical tube. The final volume is adjusted to 50 mL with room temperature PBS (without calcium and magnesium). After washing twice in PBS, the cells are resuspended in complete CTS media (Gibco, #A1048501; #A10484-02).

PBMCs or THP-1 cells or CHO cells expressing FcγRIIB (40,000 cells/well) are plated in 100 μl to a white 96-well plate (Corning, #3917). Two μg/mL of anti-human CD3 antibody (OKT3) (eBioscience. #16-0037) and treatment antibodies (peresolimab or isotype control antibody, for example) at final concentration of 20 μg/mL or a five-fold 7-point antibody titration starting at 10 μg/mL are added onto the PBMCs or THP-1 cells or CHO cells transfected with FcγRIIB. The cell and antibody mixtures are incubated at 37° C., 5% CO₂ for 30 minutes. NFAT-luciferase Jurkat reporter cells expressing PD-1 (50,000 cells/well) are added onto the PBMCs, THP-1 cells or CHO cells transfected with FcγRIIB, CD3 antibody and treatment antibody mixtures on 96-well plates, and incubated at 37° C., 5% CO₂ for 5 hours. At the end of the incubation, the plates are brought to room temperature for 10 minutes. Eighty (80) microliters of BioGlo reagent (Promega, #G7940) are added to the wells, and incubated for 10 minutes at room temperature. Luminescence is read by LMaxII384 (Molecular Devices) or Envision 2105 (Perkin Elmer).

It was previously reported (see, for example, U.S. Pat. No. 10,493,148), based on experiments performed essentially as described above, that peresolimab (i.e., Antibody 1 in U.S. Pat. No. 10,493,148) inhibited human PD-1 NFAT-luciferase reporter cell activity with an average IC₅₀ from four independent experiments of 75.2+/−37.6 ng/ml whereas isotype control antibody did not significantly inhibit NFAT signaling. These data demonstrated that peresolimab was able to inhibit T cell receptor activation induced NFAT signaling. In addition, we assessed three series of human PD-1 mAbs to further test the epitope effect. Antigen binding domain E is the same as in pemprolizumab which blocks ligand binding, while antigen binding domain series F and G are humanized rabbit mAbs binding to similar but not identical epitopes on human PD-1 ECD which do not block PDL1 binding. The G series does not block PDL2 binding either, whereas the F series partially blocks PDL2. The three antigen binding domains were paired with Fc regions as described in Table 5.

TABLE 5
Human PD-1 antibodies used in cell-based T cell activation inhibition assays

| Antigen | | | PD-1 | | |
|---|---|---|---|---|---|
| binding | | | KD | | Known effect |
| domain | PDL1 | PDL2 | (nM)¹ | Human Fc² | of Fc mutation |
| E | Blocking | Blocking | 9.7 | IgG1 | — |
| F1 | Non- | Partial | 1.4 | IgG1 | — |
| | blocking | blocking | | IgG1 L234A + L235E + G237A + | Minimal FcγR |
| | | | | A330S + P331S | binding |
| G1 | Non- | Non- | 7.5 | IgG1 | — |
| | blocking | blocking | | IgG4 S228P + F234A + L235A | Minimal FcγR |
| | | | | (IgG4PAA)⁴ | binding except |
| | | | | | FcγRI |
| G2 | | | 2.3 | IgG1 | — |
| | | | | IgG1 L234A + L235E + G237A + | Minimal FcγR |
| | | | | A330S + P331S (IgG1EN)³ | binding |
| | | | | IgG4 S228P + F234A + L235A | Minimal FcγR |
| | | | | (IgG4PAA)⁴ | binding except |
| | | | | | FcγRI |

¹Affinity to human PD-1 ECD measured by SPR method at 37° C.
²Fc mutations identified by EU numbering system.
³see, for example, Gross, J.A., Immunity, 15(2): 289-302 (2001)
⁴see, for example, Angal, S., Mol. Immunol., 30(1): 105-08 (1993)

All three PD-1 mAbs, E, F1, and G1 in the context of human IgG1, were able to inhibit TCR activation upon CD3 antibody activation as witnessed by reduction on NFAT signaling in a PD-1 Jurkat NFAT reporter cells assay (data not shown), whereas a human IgG1 isotype control antibody was not able to do so. However, when the same antigen binding domains were incorporated into mAbs with a non- or much reduced-FcγR engaging Fc such as IgG1 L234A+L235E+G237A+A330S+P331S and IgG4 S228P+F234A+L235A, they failed to show activity in this assay. Clearly, the activity is dependent on the ability of the antibodies to bind to the FcγRs regardless of the PD-1 epitope. This data agrees with a different assay, a bead-based assay, that indicated PD-1 agonism can occur when PD-1 is brought into the proximity with the activated immune receptor regardless of the PD-1 epitope (data not shown).

Similar results were obtained when PBMCs or FcγRIIB expressing CHO cells were replaced by other cell lines expressing FcγRs, including THP-1 cells, Raji cells or various FcγR transfected CHO cells (data not shown), suggesting that the agonist activity was not specific to the FcγRIIB transfected CHO cell line or PBMCs but the result of co-clustering of PD-1 and TCR driven by anchoring CD3 and PD-1 mAbs to the same microdomains by virtue of them binding to FcγRs on the surface of the T cell stimulating cell. The ability of the PD-1 mAbs to inhibit T cell activation in the bead-based assay in the absence of any FcγRs, on the other hand, corroborates that the driver of the inhibitory activity is not intrinsic FcγR activity but due to physical clustering of PD-1 with the TCR.

### Example 5: PD-1 Antibody Fc Interactions with FcγRs Drive PD-1 Agonist Activity in a Physiological TCR-MHC Stimulation Context

The ability of the certain PD-1 antibodies to support PD-1 activity in the context of physiological Major Histocompatibility Complex (MHC) stimulation was also assessed. MHC based stimulation is critical for therapeutic translation, as in the physiological MHC stimulation molecules can move freely on the T cell and antigen presenting cell surfaces to orchestrate molecular interactions and appropriate molecular placements in time and in place. We assessed the ability of PD-1 Abs to inhibit T cell activation induced by various MHC based stimulation methods, including antigen recall, allogenic response and superantigen stimulation.

### Superantigen Stimulation

Superantigens bridge MHCII molecules on the APC with specific TCR Vβ chains on T cells allowing a robust oligoclonal T cell activation (Irwin, M. J. and Gascoigne, N. R. (1993) J Leukoc Biol. 54:495-503). Essentially as described previously, bacterial superantigen Staphylococcal enterotoxin B (SEB) was used to enable robust assessment of T cell activity in a setting where the TCR and MHC can freely interact and re-localize on the cellular surface. Human CFSE labelled PBMCs were incubated in the presence of SEB for three days and proliferation upon a titration of treatment mAb was quantitated by assessing the number of CFSE diluted CD4 T cells with reduced CFSE staining was quantitated using flow cytometry. Results discussed below were representative from at least four experiments.

### Allogenic T Cell DC Response

The ability of the PD-1 agonist mAbs to inhibit T cell activation in an MHC based stimulation context was also assessed using a mixed leucocyte reaction (MLR) assay. In the MLR study, T cells and dendritic cells (DCs) are sourced from different individuals, because of an MHC mismatch between the T cell and DC donor, some T cells will express a TCR which can recognize the foreign MHC on the DC inducing T cell activation. More specifically, DCs were differentiated from human PBMC isolated monocytes by a seven-day culture in the presence of hIL-4 and hGM-CSF. The DCs were then cocultured with CD4 T cells isolated from human PBMCs for four days with 30 g/mL of treatment antibodies in the presence or absence of 10 μg/mL blocking PDL1/2 mAbs. T cell proliferation was assessed after four days by uptake of [3H] thymidine over the last 18 hours of culture. Results were analyzed as % proliferation inhibition as compared to non-treated samples. The results discussed below are representative of at least three experiments. The three antibodies tested in the MLR experiments were G2 IgG1, F1 IgG1, and F1 IgG1 L234A+L235E+G237A+A330S+P331S (i.e., F1 IgG1EN).

### Antigen Recall

An antigen recall response assay employing a cocktail of a HLAII restricted T cell epitope peptide pool derived from cytomegaly virus (CMV), Epstein Barr virus (EBV), influenza and tetanus to stimulate PBMCs was used to further assess physiological peptide-MHC (pMHC) stimulation. More specifically, human PBMCs were stimulated for five days in the presence of a HLAII restricted T cell epitope peptide pool derived from CMV, EBV, influenza and tetanus in the presence of 10 μg/ml PD-L1/2 Abs and 30 μg/ml of treatment mAbs. Proliferation was quantitated using [3H] Thymidine uptake over the last 18 hours. Results summarized below are representative of at least six experiments. F1 IgG1 and F1 IgG1EN mAbs were tested in this antigen recall study.

Briefly stated, both mAb F1 and G2 with human IgG1 inhibit T cell activation in the context of various MHC stimulation methods. G2 IgG1 and F1 IgG1 mAbs inhibit SEB stimulation induced T cell proliferation (see, Tables 6A and 6B), while F1 IgG1 mAb inhibits T cell proliferation in the antigen recall assay and in mixed lymphocyte reaction (data not shown). This activity was clearly FcγR binding dependent as demonstrated by the lack of bioactivity of antibodies with the same PD-1 antigen binding domain with an Fc that did not engage with FcγRs, such as F1 hIgG1 L234A+L235E+G237A+A330S+P331S (see, Table 6A). Abatacept (CTLA4-Fc), an established therapeutic, which blocks CD28 costimulation, was used as positive control in antigen recall experiments. Thus, both F and G series mAbs with human IgG1 inhibited T cell activation in the context of various MHC stimulation methods whereas the same antigen binding domains in the context of a non-FcγR engaging Fc or IgG1 isotype control mAb were not able to do so.

In conclusion, FcγR interactions are critically important in driving PD-1 agonist activity in the context of physiological MHC stimulation.

TABLE 6A and 6B
Fc dependency in SEB proliferation assay for mAbs with (A)
Antigen Binding Domain F and (B) Antigen Binding Domain G2

| | | % T cell | | % PD-1 | | |
|---|---|---|---|---|---|---|
| Antigen | | Proliferation | | Expression | | |
| Binding | | Inhibition | | Inhibition | | |
| Domain | Human Fc | Mean | SEM | Mean | SEM | Number |

A.

| | | | | | | |
|---|---|---|---|---|---|---|
| Isotype | IgG1 | 8.90 | 5.7 | −4.1 | 6.1 | 6 |
| control | | | | | | |
| F1 | IgG1 | 40.30 | 5.5 | 50 | 2.2 | 6 |
| | IgG1 K322A | 39.80 | 2.1 | 47.2 | 2.3 | 6 |
| | IgG1 L234A + | 6.70 | 2.4 | −11.1 | 5.9 | 6 |
| | L235E + G237A + | | | | | |
| | A330S + P331S | | | | | |
| | IgG4 S228P + | 8.50 | 4.3 | 5.5 | 4.8 | 6 |
| | F234A + L235A | | | | | |
| | IgG1 S267E + | 1.80 | 2.8 | 48.7 | 2 | 6 |
| | L328F | | | | | |
| | IgG1 G237 + | 5.00 | 2.2 | 21.8 | 3.5 | 6 |
| | H268D + P271G + | | | | | |
| | A330R | | | | | |
| | IgG1 S239D + | 92.50 | 3.4 | 57.9 | 2.7 | 6 |
| | I332E | | | | | |
| Abatacept | | 14.30 | 3.6 | −2.5 | 4.4 | 6 |

B.

| | | | | | | |
|---|---|---|---|---|---|---|
| Isotype | IgG1 | 3.75 | 6.34 | −2.07 | 2.11 | 10 |
| control | | | | | | |
| G2 | IgG1 | 56.46 | 6.85 | 50.78 | 4.09 | 10 |
| | IgG1 K322A | 58.30 | 7.05 | 45.71 | 3.80 | 10 |
| | IgG1 L234A + | 10.89 | 9.40 | 16.24 | 5.83 | 8 |
| | L235E + G237A + | | | | | |
| | A330S + P331S | | | | | |
| | IgG1 Q295A | 30.96 | 8.51 | 17.15 | 4.49 | 10 |
| | IgG4 S228P | 17.98 | 7.84 | 13.65 | 4.55 | 10 |
| | IgG1 R292A | 57.90 | 7.41 | 44.80 | 3.66 | 10 |
| | IgG1 S267A | 51.20 | 6.73 | 42.17 | 3.17 | 10 |
| | IgG1 N325S + | 6.17 | 7.51 | 16.08 | 3.24 | 10 |
| | L328F | | | | | |
| | IgG1 P238D | 5.94 | 6.72 | 4.53 | 3.77 | 10 |
| | IgG1 G236D | 9.78 | 7.42 | 0.06 | 3.39 | 10 |
| | IgG1 S239D + | 95.60 | 2.90 | 75.10 | 2.30 | 10 |
| | I332E | | | | | |
| Abatacept | | 16.24 | 5.15 | 16.43 | 2.24 | 10 |

### Example 6: Potential Effector Functions of PD-1 IgG1 mAbs

It is well known that human IgG1 mAb interactions with FcγR or C1q may induce effector functions, namely antibody-dependent cellular cytotoxicity (ADCC) and complement-dependent cytotoxicity (CDC), depending on factors such as the antigen binding domain affinity, the target, or the target expression level.

The potential of the PD-1 IgG1 mAbs to trigger effector functions and kill T-cells was assessed. More specifically F1 and G2 IgG1 mAbs and variants thereof with a K322A Fc region, which is known to completely abrogate complement binding (see, Hezareh, M., J. Virol., 75 (24): 12161-12168 (December 2001)), were tested in these experiments. Briefly stated, G2 IgG1 mAb was not able to drive CDC activity (data not shown). The lack of CDC contribution to the bioactivity was also illustrated by similar agonist activity between IgG1 and IgG1 K322A mutant for both F1 and G2 mAbs (see, Table 6A and Table 6B). Furthermore, G2 IgG1 mAb did not show NK cell driven ADCC on peripheral blood T cells (see, Table 9). However, when T cells were activated to induce PD-1 expression, prior to the NK cell assay, G2 IgG1 mAb demonstrated modest ADCC activity (see, Table 9). This data suggests that the PD-1 IgG1 mAb has some potential for a two-pronged mechanism of action, agonizing PD-1 and ADCC, to reduce activated T cells when PD-1 is highly expressed.

### Example 7: PD-1 FcγR Engaging Antibodies Drive Bioactivity In Vivo

Because neither antigen binding domain F nor G series bind to mouse PD-1, a well-known humanized GvHD model for in vivo studies was used to confirm that PD-1 agonist mAb activity was FcγR dependent in vivo, as well as in vitro. Briefly stated, human PBMCs are transferred to an immunodeficient NSG mice and human T cells are activated due to mouse peptides presented on human immune cells or due to a xenogeneic MHC mismatch resulting in a graft versus host disease (GvHD) as manifested by body weight loss over time. More specifically, GvHD was induced by engrafting 12.5 million human PBMCs intravenously into immunodeficient NSG (NOD·Cg-Prkdscid Il2rgtm 1 Wjl/SzJ) mice. Mice were treated with 10 mg/kg of treatment mAbs subcutaneously starting on day 0, twice weekly for the remainder of the experiment. Mice were weighted 2-3 times a week, and the weight was used as a surrogate for development of GvHD. Body weight change was calculated as a percentage of the baseline weight on day 0. All mice were euthanized when most isotype control mice had progressed to disease. Blood was collected for plasma cytokine analysis by Mesoscale Discovery (MSD) platform and spleens were collected for flow cytometry analysis for T regulatory cells and T effector cells. Results discussed are based on average+/−SEM % body weight as compared to baseline from 8-9 mice.

Both human and mouse FcγR engaging mAbs were tested in this model for their ability to protect mice from GvHD. Mouse IgG1 engages with some human FcγRs and strongly with some mouse FcγRs, including FcγRIIB and FcγRIII (see, for example, Wang, et al., Eur. J. Immunol., 52:753-759 (2022)). Therefore, we compared the efficacy of F1 human IgG1 mAb and F1 mouse IgG1 mAb. In addition, we assessed the poorly FcγR engaging G1 and F2 human IgGPAA mAbs (note: the F2 antigen binding domain differs from F1 by two amino acid residues with a comparable PD-1 binding affinity as F1) with their mouse IgG1 counterparts, G1 mouse IgG1 and F2 mouse IgG1 in this model.

Experiments performed essentially as described in this Example, clearly showed that only those antibodies capable of engaging human FcγRs, both human IgG1 and mouse IgG1 (Overdjik 2012 and Wang 2022), were able to attenuate disease progression as measured by body weight loss comparing F1 human IgG1 and F1 mouse IgG1 mAbs with the respective isotype control mAbs. To the contrary, the poorly FcγR engaging antibodies such as G1 human IgG4PAA and F2 human IgG4PAA mAbs, as compared to the respective isotype control mAbs, were not able to do so. Additionally, the FcγR engaging antibodies inhibited the increase in plasma human Th1 (IFNγ) and Th2 (IL-13) inflammatory cytokines associated with disease progression and reduced effector T cells (data not shown). In addition, the efficacy was associated with increases in percentage of T regulatory cells. The increased % Treg difference between the FcγR engaging (mIgG1) and poorly engaging (hIgG4PAA) was statistically significant for both mAbs with G1 and F2 antigen binding domains (data not shown). Adalimumab (a TNF blocking mAb), an established therapeutic mAb for the treatment of autoimmune diseases, was used as a positive control in some of the experiments. These in vivo experiments further support the surprisingly important role for FcγR engagement to drive PD-1 mAb agonist activity.

### Example 8: Higher PD-1 Affinity Correlates with More PD-1 Agonism

While antagonist mAbs typically work better if they have a higher affinity to completely block ligand binding to the receptors, it remains unpredictable for agonist mAbs whether the antibody will be more efficacious if they have a higher affinity. Indeed, for some agonist antibodies, an inverse correlation has been shown (Chodorge M 2012; Yu 2022). This might be explained by the higher probability of low affinity mAbs, as compared to higher affinity mAbs, to bind with both antigen binding domains simultaneously leading to clustering of the receptors, which may drive activation of some receptors.

To assess how PD-1 affinity affects agonist activity, several human PD-1 IgG1 mAbs with structurally similar human PD-1 ECD antigen binding domains with a range of affinities to human PD-1 ECD (as measured by Biacore at 25° C.) were generated (see, Table 7).

TABLE 7
PD-1 agonist mAbs of different affinities from the G epitope series

| G Antigen | PD-1 | PD-1 | PD-1 | T cell |
|---|---|---|---|---|
| binding | kon | Koff | K_{D} | inhibition: |
| domains | (1/Ms) | (1/s) | (nM) | IC₅₀ (μg/mL) |
| A | 2.0 × 10⁵ | 1.2 × 10⁻⁴ | 0.6 | 0.014 |
| B | 1.7 × 10⁵ | 4.6 × 10⁻⁴ | 2.7 | 0.04 |
| C | 7.6 × 10⁴ | 3.6 × 10⁻³ | 48 | 0.24 |
| D | 4.7 × 10⁴ | 5.2 × 10⁻³ | 111 | 0.78 |

These mAbs (i.e., A-D as shown in Table 7) were compared to assess their ability to inhibit T cell activation using the SEB stimulation assay. More specifically, human CFSE labelled PBMCs were incubated in the presence of SEB 30 μg of each of the human IgG1 PD-1 mAbs A-D for three days, and proliferation upon mAb treatment was quantitated by assessing the number of CD4 T cells with reduced CFSE staining by using flow cytometry. The half-maximal inhibitory concentration (IC₅₀) for each PD-1 affinity variant is shown. Data are representative of two independent experiments.

The results shown in Table 7 illustrate that PD-1 agonist activity correlates to PD-1 binding affinity in this range, with the higher affinity PD-1 mAbs resulting in higher bioactivity.

### Example 9: Multiple FcγR Interactions Contribute to PD-1 Agonist Activity

A panel of human Fc variants in the context of F1 and G2 antigen binding domains were constructed and then their binding to various FcγRs by Biacore (see, Table 8) as well as their bioactivity in a SEB proliferation assay was assessed in order to understand which FcγRs are the most critical for the inhibitory function mediated by PD-1 mAbs.

Briefly described, human PBMCs were stimulated for three days in the presence of SEB and 30 μg/mL of treatment mAbs in triplicates. The number of CD4 T cells with reduced CFSE staining was quantitated using flow cytometry. In addition, geometric mean fluorescent intensity was quantitated by PD-1 Ab staining using flow cytometry. Results shown are % T cell proliferation compared to untreated samples, mean+/−SEM from 6 donors (see, Table 6A) or 8-10 donors (see, Table 6B).

Surprisingly, the PD-1 mAbs with different Fc variants showed a range of activities in this assay. The variants that inhibited T cell activation showed measurable binding to both FcγRIIA and FcγRIIIA. Despite enhanced FcγRIIB binding, F1 IgG1 S267E/L328F and F1 G237D/H268D/P271G/A330R inhibited T cell proliferation poorly. However, these variants were able to inhibit PD-1 expression better than T cell proliferation. For the G2 Fc variants, both IgG1 R292A and IgG1 Q295A exhibited barely measurable binding to FcγRIIB while maintaining similar affinity to the other FcγRs compared with IgG1 but showed significant inhibitory activity. The IgG1 S239D/I332E mAbs had enhanced binding affinity to all FcγRs and demonstrated strong inhibitory activity. The IgG4P variant had noticeable loss in affinity to FcgRIII and FcgRII and exhibited reduced inhibitory activity compared to IgG1. Taken together, these data surprisingly suggest that multiple FcgR interactions contribute to PD-1 agonist activity.

Additional G2 IgG1 mAb Fc variants (Table 8) were constructed and their bioactivity in the SEB assay, in ADCC assays with T cells that were activated in vitro to induce PD-1 expression, and in ADCC assays with blood circulating T cells in PBMCs was tested (Table 9). More specifically, in the SEB assay human PBMCs were stimulated for three days in the presence of SEB and a concentration range of mAbs from 0.4 ng/ml to 100 μg/mL with 8-fold dilutions. Number of CD4 T cells with reduced CFSE staining was quantitated using flow cytometry. Results shown are average % T cell proliferation inhibition compared to untreated samples from 5 donors.

ADCC was assessed by freshly isolated NK cells from both ConA activated T cells and from freshly isolated PBMCs with a concentration range of mAbs from 0.4 ng/ml to 100 mg/mL with 8-fold dilutions. Number of live CD4 T cells were quantitated by flow cytometry. Results shown are mean+/−SEM % CD4 T cell killing compared to untreated samples from 8 (for activated T cells) or 3 (from freshly isolated PBMCs) donors. The results shown in Table 8 revealed that increased FcγR binding increased inhibition of T cell proliferation. However, increased FcγRIIIA affinity also increased ADCC activity, suggesting that with enhanced FcγRIIIA binding the response mechanistically may shift from agonism towards ADCC activity.

TABLE 8
Human IgG Fc variants binding affinity to human FcγRs by Biacore at 25° C.

| Antigen | | | | | | | | | | | | | |
|---|---|---|---|---|---|---|---|---|---|---|---|---|---|
| binding | Human | | | FcγRIIa | | FcγRIIa | | | | FcγRIIIa | | FcγRIIIa | |
| domain | Fc¹ | FcγRI | | 131H | | 131R | | FcγRIIb | | 158F | | 158V | |
| F1 | IgG1 | 55 | pM | 910 | nM | 1.45 | μM | 5.23 | μM | 1.7 | μM | 342 | nM |
| F1 | IgG1 L234A + | No | | No | | >10 | μM | >10 | μM | No | | No | |
| | L235E + G237A + | binding | | binding | | | | | | binding | | binding | |
| | A330S + P331S | | | | | | | | | | | | |
| F1 | IgG1 S267E + | 15 | pM | 2 | μM | 8.33 | nM | 22.3 | nM | 4.2 | μM | 7.7 | μM |
| | L328F | | | | | | | | | | | | |
| F1 | IgG1 G237D + | 16.8 | nM | >10 | μM | 360 | nM | 40 | nM | >10 | μM | >10 | μM |
| | H268D + P271G + | | | | | | | | | | | | |
| | A330R | | | | | | | | | | | | |
| F1 | IgG1 S239D + | 7.54 | pM | 1.2 | μM | 215 | nM | 0.37 | μM | 37 | nM | 16.4 | nM |
| | I332E | | | | | | | | | | | | |
| F1 | IgG1 | 49 | pM | 1.16 | μM | 970 | nM | 3.6 | μM | 1.69 | μM | 357 | nM |
| | K322A | | | | | | | | | | | | |
| F1 | IgG4 S228P + | 87.4 | nM | >10 | μM | >10 | μM | >10 | μM | >10 | μM | 9.2 | μM |
| | F234A + L235A | | | | | | | | | | | | |
| G2 | IgG1 | 89 ± 16 | pM | 693 ± 55 | nM | 802 ± 210 | nM | 4.9 ± 0.7 | μM | 1.2 ± 0.2 | μM | 106 ± 67 | nM |
| G2 | IgG1 L234A + | No | | No | | >10 | μM | >10 | μM | No | | No | |
| | L235E + G237A + | binding | | binding | | | | | | binding | | binding | |
| | A330S + P331S | | | | | | | | | | | | |
| G2 | IgG1 | 71 ± 10 | pM | 931 ± 93 | nM | 639 ± 192 | nM | 4.1 ± 0.6 | μM | 1.2 ± 0.2 | μM | 106 ± 53 | nM |
| | K322A | | | | | | | | | | | | |
| G2 | IgG4 | 667 ± 272 | pM | 4.3 ± 0.3 | μM | 2.0 ± 0.5 | μM | 3.4 ± 0.5 | μM | >10 | μM | 2.5 ± 1.6 | μM |
| | S228P | | | | | | | | | | | | |
| G2 | IgG1 | 7 ± 2 | nM | >10 | μM | >10 | μM | 1.4 ± 0.2 | μM | >10 | μM | >10 | μM |
| | P238D | | | | | | | | | | | | |
| G2 | IgG1 S239D + | 14 ± 3 | pM | 199 ± 28 | nM | 133 ± 37 | nM | 394 ± 72 | nM | 36 ± 4 | nM | 12 ± 3 | nM |
| | I332E | | | | | | | | | | | | |
| G2 | IgG1 N235S + | 127.3 | pM | 2.59 | μM | 128 | nM | 450 | nM | 6.22 | μM | 7.54 | μM |
| | L328F | | | | | | | | | | | | |
| G2 | IgG1 | 8.9 | nM | 1.1 | μM | 2.0 | μM | 1.8 | μM | 4.4 | μM | 4.4 | μM |
| | G236D | | | | | | | | | | | | |
| G2 | IgG1 | 55 ± 7 | pM | 547 ± 104 | nM | 0.6 ± 1 | μM | 2.0 ± 2.5 | μM | 1.0 ± 0.7 | μM | 109 ± 129 | nM |
| | S267A | | | | | | | | | | | | |
| G2 | IgG1 | 107 ± 17 | pM | 965 ± 228 | nM | 2.5 ±0.9 | μM | >10 | μM | 1.1 ± 0.2 | μM | 112 ± 36 | nM |
| | R292A | | | | | | | | | | | | |
| G2 | IgG1 | 108 ± 18 | pM | 899 ± 25 | nM | 2.2 ± 1.5 | μM | >10 | μM | 1.7 ± 0.3 | μM | 146 ± 76 | nM |
| | Q295A | | | | | | | | | | | | |
| G2 | IgG1 P247I + | 56 ± 8 | pM | 372 ± 9 | nM | 358 ± 21 | nM | 2.3 ± 0.3 | μM | 511 ± 110 | nM | 47 ± 12 | nM |
| | A339Q | | | | | | | | | | | | |
| G2 | IgG1 G236A + | 21 ± 4 | pM | 40 ± 1 | nM | 33 ± 3 | nM | 504 ± 65 | nM | 39 ± 4 | nM | 16.8 ± 0.5 | nM |
| | S239D + I332E | | | | | | | | | | | | |
| G2 | IgG1 G236A + | 18 ± 6 | pM | 103 ± 2 | nM | 79 ± 6 | nM | 1.1 ± 0.2 | μM | 24 ± 2 | nM | 13.5 ± 0.2 | nM |
| | S239D + I332E + | | | | | | | | | | | | |
| | A330L | | | | | | | | | | | | |
| G2 | IgG1 S298A + | 43 ± 7 | pM | 5.0 ± 0.3 | μM | 3.3 ± 0.2 | μM | >10 | μM | 203 ± 49 | nM | 68 ± 3 | nM |
| | E333A + K334A | | | | | | | | | | | | |
| G2 | IgG1 F243L + | 36 ± 8 | pM | 171 ± 8 | nM | 378 ± 24 | nM | 2.2 ± 0.3 | μM | 95 ± 21 | nM | 64 ± 4 | nM |
| | R292P + Y300L + | | | | | | | | | | | | |
| | V305I + P396L | | | | | | | | | | | | |
| G2 | IgG1 S239D + | 14 ± 4 | pM | 593 ± 66 | nM | 348 ±34 | nM | 1.0 ± 0.2 | μM | 21 ± 1 | nM | 11.6 ± 0.8 | nM |
| | I332E + A330L | | | | | | | | | | | | |

¹Fc mutations identified by EU numbering system.

TABLE 9
G2 IgG1 mAb Fc variants with high affinity
to FcγR increase bioactivity and ADCC

| | | | % ADCC | | | |
|---|---|---|---|---|---|---|
| G2 | % Inhibition | | Activated | | % ADCC | |
| Human IgG1 | Proliferation | | T cells | | PBMC | |
| Fc region | Mean | SEM | Mean | SEM | Mean | SEM |
| Isotype control | 0 | 3.1 | −0.7 | 0.2 | −0.6 | 0.2 |
| WT | 36.9 | 4.3 | 3.3 | 0.6 | 0.2 | 0.04 |
| P247I + A339Q | 51.5 | 5.2 | 4.9 | 0.6 | 1.6 | 0.5 |
| S239D + I332E + | 76.1 | 2.4 | 18.6 | 1.5 | 7.7 | 2.2 |
| A330L | | | | | | |
| S239D + I332E | 72.4 | 2.4 | 15.9 | 1.2 | 6.3 | 1.7 |
| G236A + S239D + | 70.2 | 3.1 | 14 | 1.3 | 5.9 | 1.6 |
| I332E | | | | | | |
| F243L + R292P + | 71.5 | 3.1 | 18.2 | 1.2 | 7.3 | 2 |
| Y300L + V3051 + | | | | | | |
| P396L | | | | | | |
| G236A + S239D + | 73.9 | 1.6 | 20.6 | 1.2 | 8.3 | 2.4 |
| I332E + A330L | | | | | | |
| S298A + E333A + | 73.2 | 2.4 | 8.9 | 1.0 | 3.3 | 1 |
| K334A | | | | | | |

As shown herein, the Fc region of the PD-1 mAbs and its interactions or the lack of with various FcγRs plays an important role in defining the ultimate functional outcome and mechanism of action for therapeutic PD-1 mAbs. PD-1 agonism can be achieved therapeutically by leveraging mAb Fc-FcγR engagement to co-localize TCR with PD-1 while PD-1 mAb induced clustering is unlikely to be a driver for PD-1 agonism. The interactions between PD-1 mAb and multiple FcγRs are critical in driving PD-1 agonist activity regardless of the epitopes, and that the bioactivity can be fine-tuned by altering the mAb's Fc affinity to various FcγRs as well as by the antigen binding domain's affinity to PD-1.

Human FcγRs have emerged as an important contributor to bioactivity of various therapeutic mAbs (see, e.g., White, A. L., Cancer Immunol Immunother. 62:941-948 (2013)), and a special role for the inhibitory FcγRIIB has been identified for some costimulatory mAbs for immunological applications (see, e.g., Li, F., et al., Science. 333:1030-1034 (2011); Li, F., et al., Proc Natl Acad Sci USA. 109:10966-10971 (2012); White, A. L., J Immunol. 187:1754-1763 (2011)). An important driver of the FcγRIIB mediated agonistic activity has been suggested to be clustering mediated receptor activation. However, we provide ample evidence that PD-1 clustering itself surprisingly may not play an important role for PD-1 antibody mediated agonism. More specifically, we found that a single binding arm or a monovalent PD-1 mAb is at least as efficacious as a two-arm binding traditional bivalent PD-1 mAb (data not shown). In addition, we found no activity with higher order multimeric Abs, such as a tetravalent PD-1 mAb (data not shown) in the context of human IgG4PAA, further suggesting that clustering alone is not sufficient to drive PD-1 agonism. Having demonstrated the importance of PD-1 redistribution on the cellular membrane to the close proximity of the TCR, it seems that the FcγRs work as a hook in the IS to allow PD-1 to relocate to a close proximity of the TCR to enable inhibition of TCR signaling.

Receptor localization on the cellular membrane, to domains with for instance kinase or phosphatase activity, is thought to be important in driving activation and inhibition of receptors in the immunological synapse (IS). However, receptor re-localization on the membrane has not yet been described previously to explain FcγR dependent mAb agonism. FcγRs could drive relocation of the mAb bound target receptor based on the FcγR localization domain on the cellular surface. FcγR binding of the mAb-target complex may also play an important role in driving size mediated location in the IS. Segregation of intercellular receptor-ligand complexes that span different lengths has been suggested to occur in the IS. Therefore, the size of the interacting ectodomains of FcγR-PD-1 mAb-PD-1 interacting complex may be an important driver for the PD-1 location next to the TCR-MHC complex, which is estimated to occupy a length of about 15 nM in the IS (see, e.g., Dustin, M. L., Arthritis Res. 4 Suppl 3: S119-125 (2002)).

As disclosed herein, we have elucidated the importance of each FcγR in driving PD-1 agonism by generating a panel of Fc variants with varying affinity to FcγRs. More specifically, wild type human IgG1 binds to most FcγRs with high nanomolar or low micromolar affinity except picomolar affinity to FcγRI. All the Fc variants showing robust bioactivity exhibited strong binding to FcγRI. However, not all the Fc variants with strong FcγRI binding, such as G2 IgG1 N235S/L328F mAb, show bioactivity. Therefore, FcγRI surprisingly does not play a critical role in supporting PD-1 agonist activity. In retrospect, FcγRI in PBMCs is likely saturated with endogenous IgG1 antibodies present in adult blood at a high concentration range of 3.76-10.6 mg/mL (Rasmussen, K. F., Immun Inflamm Dis. 9:1009-1015 (2021)) due to their strong FcγRI affinity. Perhaps, the lower binding affinity of endogenous antibodies to the other FcγRs, FcγRs, FcγRIIA, FcγRIIB and FcγRIIIA, on the other hand, is less likely to prevent these FcγRs to bind to exogeneous mAbs. Their availability appears very important in driving the mAb interaction for a PD-1 agonist mAb in the IS, where mAb binding to PD-1 can then position the mAb Fc region in the two-dimensional surface to induce avidity driven interactions with the FcγRs expressed on the APC. Overall, our assessment of PD-1 mAb Fc variants demonstrate that bioactivity correlates better with FcγRIIIA binding than with FcγRIIA binding. In particular, G2 IgG1 G236D and G2 IgG1 N235S/L328F variants, both poor binders of FcγRIIIA, showed lower activity than would be expected based on their FcγRIIA affinity (Table 6B) if FcγRIIA were the main driver of the bioactivity. Several Fc variants in our experiments suggested that FcγRIIB binding alone is not a significant driver of IgG1 mediated bioactivity. For example, human IgG4 S228P binds to most FcγRs significantly weaker than human IgG1 except for FcγRIIB. Yet G2 IgG4 S228P mAb exhibited significantly lower ability to inhibit T cell activation (Table 6B) than G2 IgG1 mAb. We also assessed FcγRIIB affinity enhanced Fc variants for their ability to drive agonistic activity, including G2 IgG1 N325S/L328F, F1 IgG1 S267E/L328F, and F1 IgG1 G237D/H268D/P271G/A330R mAbs (Table 6A and Table 6B). While some bioactivity was observed with these Fc variants, none of the mAbs elicited strong bioactivity in the PBMC based T cell proliferation assay (Table 6A, B), refuting the importance of FcγRIIB binding alone.

Taken together, our data surprisingly suggests that at least in the context of IgG1, FcγRIIIA is the most important FcγR driving PD-1 antibody mediated bioactivity. FcγRIIA may also contribute to the activity, whereas FcγRI and FcγRIIB may play a minor role. While our conclusions agreed with the earlier report by Suzuki, et al. for the important role of FcγR interactions in driving PD-1 agonistic activity, our findings differ from theirs regarding the type of FcγR and PD-1 epitopes that are important to PD-1 agonist activity (see, Suzuki, K., et al., Sci. Immunol. 8 (79), (2023)). We conclude that multiple FcγR interactions drive PD-1 agonist activity with FcγRIIIA playing an important role, whereas Suzuki et al suggested that FcγRIIB binding was critically important. This discrepancy may be explained by differences in the FcγR dominance of their assay system and in the PD-1 Fc variants studied in their studies. Suzuki et al studied the agonism in the context of cells that only express FcγRIIB or highly express FcγRIIB in combination with PD-1 Fc variants with enhanced binding to FcγRIIB, while we relied on human PBMC for most experiments.

We also showed that FcγR interactions could induce agonist activity in PD-1 mAbs of various epitopes including ligand blocking epitope. This contrasts with the report by Suzuki et al which suggested that recognition of membrane-proximal extracellular region is important in driving agonist activity. In addition, our finding that higher PD-1 affinity correlates with more bioactivity contrasts with a recently published paper, which suggested that reduced affinity to PD-1 boosts PD-1 agonism (Yu, X., et al., Nature. 614:539-547 (2023)). Yu, et al. reported that low affinity nivolumab variants demonstrated stronger PD-1 clustering and stronger T cell suppression than nivolumab in an NFAT-reporter cell assay, where the NFAT activity was induced by capturing both CD3 mAb and the PD-1 affinity variants onto a CHO cell expressing an anti-IgG scFv. It is likely that co-localization of PD-1 and TCR by virtue of their respective antibodies binding to the anti-IgG on the cell surface overdrives the need for a soluble therapeutic antibody to intrinsically enable PD-1 and TCR co-localization. Therefore, their findings may not as readily translate to therapeutic outcomes compared with more physiological MHC stimulation methods.

Our finding on the role of PD-1 mAb-FcγR engagement strongly suggests that the induction of PD-1 agonist activity may lead to reduced anti-tumor efficacy of PD-1 antagonist mAbs. In our fully human PBMC based experiments, PD-1 lgG4 S228P mAbs exhibited modest agonist activity. Our data showed that when pembrolizumab antigen binding domain is paired with wild type IgG1 Fc, the resulting PD-1 mAb was able to inhibit T cell activation as measured by NFAT activity and demonstrated robust ability to inhibit T cell activation in a PBMC based SEB assay (data not shown). These data indicate that mild FcγR engagement of PD-1 antagonist mAbs may be responsible for the reduced anti-tumor activity.

We have clearly shown that PD-1 mAb binding to various FcγRs can sanction PD-1 mAbs with PD-1 agonistic activity. The agonistic activity can enable PD-1 targeting for the treatment of autoimmune diseases but is also a potential mechanism to dampen PD-1 antagonist treatments for malignant diseases. Additionally, engineering PD-1 mAb antigen binding domain affinity as well as Fc domain binding affinity to various FcγRs may lead to improved PD-1 agonist mAbs for therapeutic applications.

1.-28. (canceled)

29. An antibody that binds human programmed cell death protein 1 (human PD-1) extracellular domain (ECD) with an affinity of between about 1 pM and about 100 nM as determined by SPR at 25° C., wherein the antibody comprises a heavy chain variable region (HCVR) and a light chain variable region (LCVR), wherein the HCVR comprises heavy chain complementarity determining regions (HCDRs) HCDR1, HCDR2, and HCDR3, and the LCVR comprises light chain complementarity determining regions (LCDRs) LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least three of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA 131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., and wherein the antibody is a human PD-1 agonist.

30. The antibody of claim 29, wherein the HCVR of the antibody comprises the amino acid sequence of SEQ ID NO: 3, and the LCVR of the antibody comprises the amino acid sequence of SEQ ID NO: 4.

31. The antibody of claim 30, wherein the antibody further comprises a human IgG1 Fc region variant selected from the group consisting of:
a) a human IgG1 Fc region variant comprising P247I and A339Q; and
b) a human IgG1 Fc region variant comprising S298A, E333A, and K334A;
c) a human IgG1 Fc region variant comprising S239D and I332E;
d) a human IgG1 Fc region variant comprising S239D, I332E, and A330L;
e) a human IgG1 Fc region variant comprising G236A, S239D, and I332E; and
f) a human IgG1 Fc region variant comprising G236A, S239D, I332E, and A330L.

32. The antibody of claim 31, wherein the antibody further comprises a human IgG1 Fc region variant comprising an amino acid sequence selected from the group consisting of SEQ ID NO: 32, SEQ ID NO: 33, SEQ ID NO: 34, SEQ ID NO: 35, SEQ ID NO: 36, and SEQ ID NO: 37.

33. The antibody of claim 32, wherein the antibody further comprises a heavy chain amino acid sequence selected from the group consisting of SEQ ID NO: 38, SEQ ID NO: 39, SEQ ID NO: 40, SEQ ID NO: 41, SEQ ID NO: 42, and SEQ ID NO: 43.

34. The antibody of claim 33, wherein the antibody further comprises a light chain comprising the amino acid sequence of SEQ ID NO: 2.

35. An antibody that binds human PD-1 ECD with an affinity of between 1 pM and 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, wherein the antibody further comprises a human IgG1 Fc region variant selected from the group consisting of:
a) a human IgG1 Fc region variant comprising P247I and A339Q; and
b) a human IgG1 Fc region variant comprising S298A, E333A, and K334A;
c) a human IgG1 Fc region variant comprising S239D and I332E;
d) a human IgG1 Fc region variant comprising S239D, I332E, and A330L;
e) a human IgG1 Fc region variant comprising G236A, S239D, and I332E; and
f) a human IgG1 Fc region variant comprising G236A, S239D, I332E, and A330L, and wherein the antibody is a human PD-1 agonist.

36. The antibody of claim 35, wherein the HCVR of the antibody comprises the amino acid sequence of SEQ ID NO: 3, and the LCVR of the antibody comprises the amino acid sequence of SEQ ID NO: 4.

37. The antibody of claim 36, wherein the antibody further comprises a human IgG1 Fc region variant comprising an amino acid sequence selected from the group consisting of SEQ ID NO: 32, SEQ ID NO: 33, SEQ ID NO: 34, SEQ ID NO: 35, SEQ ID NO: 36, and SEQ ID NO: 37.

38. The antibody of claim 37, wherein the antibody further comprises a heavy chain amino acid sequence selected from the group consisting of SEQ ID NO: 38, SEQ ID NO: 39, SEQ ID NO: 40, SEQ ID NO: 41, SEQ ID NO: 42, and SEQ ID NO: 43.

39. The antibody of claim 38 which further comprises a light chain comprising the amino acid sequence of SEQ ID NO: 2.

40. A method of treating an autoinflammatory or an autoimmune disease comprising administering to a patient in need thereof an effective amount of an antibody that binds human PD-1 ECD with an affinity of between 1 pM and 100 nM as determined by SPR at 25° C., wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10, and the antibody binds at least three, or at least four of Fc [symbol] RI, Fc [symbol] RIIA_131H, Fc [symbol] RIIA_131R, Fc [symbol] RIIb, Fc [symbol] RIIIA_158F and Fc [symbol] RIIIA_158V with an affinity of:
(i) about 1 pM to about 1 μM to Fc [symbol] RI;
(ii) about 10 nM to about 10 μM to Fc [symbol] RIIA 131H;
(iii) about 1 nM to about 10 μM to Fc [symbol] RIIA_131R;
(iv) about 10 nM to about 10 μM to Fc [symbol] RIIb;
(v) about 100 pM to about 10 μM to Fc [symbol] RIIIA_158V; and
(vi) about 10 nM to about 10 μM to Fc [symbol] RIIIA_158F, respectively, as determined by SPR at 25° C., and wherein the antibody is a human PD-1 agonist.

41. The method of claim 40, wherein the HCVR of the antibody comprises the amino acid sequence of SEQ ID NO: 3, and the LCVR of the antibody comprises the amino acid sequence of SEQ ID NO: 4.

42. The method of claim 41, wherein the antibody further comprises a human IgG1 Fc region variant selected from the group consisting of:
a) a human IgG1 Fc region variant comprising P247I and A339Q; and
b) a human IgG1 Fc region variant comprising S298A, E333A, and K334A;
c) a human IgG1 Fc region variant comprising S239D and I332E;
d) a human IgG1 Fc region variant comprising S239D, I332E, and A330L;
e) a human IgG1 Fc region variant comprising G236A, S239D, and I332E; and
f) a human IgG1 Fc region variant comprising G236A, S239D, I332E, and A330L.

43. The method of claim 42, wherein the antibody further comprises a human IgG1 Fc region variant comprising an amino acid sequence selected from the group consisting of SEQ ID NO: 32, SEQ ID NO: 33, SEQ ID NO: 34, SEQ ID NO: 35, SEQ ID NO: 36, and SEQ ID NO: 37.

44. The method of claim 41, wherein the antibody further comprises a heavy chain amino acid sequence selected from the group consisting of SEQ ID NO: 38, SEQ ID NO: 39, SEQ ID NO: 40, SEQ ID NO: 41, SEQ ID NO: 42, and SEQ ID NO: 43.

45. The method of claim 44, wherein the antibody further comprises a light chain Comprising the amino acid sequence of SEQ ID NO: 2.

46. A method of treating an autoinflammatory or an autoimmune disease, comprising administering about 75 mg to about 1200 mg of an antibody that binds human PD-1 to a patient in need thereof, wherein the antibody comprises a HCVR and a LCVR, wherein the HCVR comprises heavy chain complementarity determining regions HCDR1, HCDR2, and HCDR3, and the LCVR comprises light chain complementarity determining regions LCDR1, LCDR2, and LCDR3, wherein
the HCDR1 comprises the amino acid sequence of SEQ ID NO: 5,
the HCDR2 comprises the amino acid sequence of SEQ ID NO: 6,
the HCDR3 comprises the amino acid sequence of SEQ ID NO: 7,
the LCDR1 comprises the amino acid sequence of SEQ ID NO: 8,
the LCDR2 comprises the amino acid sequence of SEQ ID NO: 9, and
the LCDR3 comprises the amino acid sequence of SEQ ID NO: 10.

47. The method of claim 46, wherein the HCVR comprises the amino acid sequence of SEQ ID NO: 3, and the LCVR comprises the amino acid sequence of SEQ ID NO: 4.

48. The method of claim 46, wherein the antibody comprises a human immunoglobulin constant region which is human IgG1.

49. The method of claim 47, wherein the antibody comprises a human immunoglobulin constant region which is human IgG1.

50. The method of claim 48, wherein the antibody comprises a heavy chain comprising the amino acid sequence of SEQ ID NO: 1 and a light chain comprising the amino acid sequence of SEQ ID NO: 2.

51. The method of claim 49, wherein the antibody comprises a heavy chain comprising the amino acid sequence of SEQ ID NO: 1 and a light chain comprising the amino acid sequence of SEQ ID NO: 2.

52. The method of claim 51, wherein the antibody comprises two heavy chains and two light chains, wherein each heavy chain comprises the amino acid sequence of SEQ ID NO: 1, and each light chain comprises the amino acid sequence of SEQ ID NO: 2.

53. The method of claim 52, wherein the antibody is peresolimab.

54. The method of claim 46, wherein the disease is rheumatoid arthritis (RA), systemic lupus erythematosus (SLE), lupus nephritis (LN), cutaneous lupus erythematosus (CLE), giant cell arteritis (GCA), vasculitis, ulcerative colitis (UC), Crohn's disease (CD), or type 1 diabetes mellitus (T1DM).

55. The method of claim 47, wherein the disease is RA, SLE, LN, CLE, GCA, vasculitis, UC, CD, or T1DM.

56. The method of claim 48, wherein the disease is RA, SLE, LN, CLE, GCA, vasculitis, UC, CD, or T1DM.

57. The method of claim 49, wherein the disease is RA, SLE, LN, CLE, GCA, vasculitis, UC, CD, or T1DM.

58. The method of claim 50, wherein the disease is RA, SLE, LN, CLE, GCA, vasculitis, UC, CD, or T1DM.

59. The method of claim 51, wherein the disease is RA, SLE, LN, CLE, GCA, vasculitis, UC, CD, or T1DM.

60. The method of claim 52, wherein the disease is RA, SLE, LN, CLE, GCA, vasculitis, UC, CD, or T1DM.

61. The method of claim 40, wherein the disease is RA, SLE, LN, CLE, GCA, vasculitis, UC, CD, or T1DM.

62. The method of claim 41, wherein the disease is RA, SLE, LN, CLE, GCA, vasculitis, UC, CD, or T1DM.

63. The method of claim 42, wherein the disease is RA, SLE, LN, CLE, GCA, vasculitis, UC, CD, or T1DM.

64. The method of claim 43, wherein the disease is RA, SLE, LN, CLE, GCA, vasculitis, UC, CD, or T1DM.

65. The method of claim 44, wherein the disease is RA, SLE, LN, CLE, GCA, vasculitis, UC, CD, or T1DM.

66. The method of claim 45, wherein the disease is RA, SLE, LN, CLE, GCA, vasculitis, UC, CD, or T1DM.

67. The method of claim 53, wherein the disease is RA, SLE, LN, CLE, GCA, vasculitis, UC, CD, or T1DM.
